(19) United States
(12) Patent Application Publication
(10) Pub. No.: US 2015/0159135 A1
(43) Pub. Date: Jun. 11, 2015

(54) Perineurium Derived Adult Stem Cells and Methods of Use

(71) Applicants: Baylor College of Medicine, Houston, TX (US); The Board of Regents of the University of Texas System, Austin, TX (US)

(72) Inventors: Elizabeth A. Davis, Missouri City, TX (US); Elizabeth A. Salisbury, Houston, TX (US); Alan R. Davis, Missouri City, TX (US); Zbigniew Gugala, Houston, TX (US)

(21) Appl. No.: 14/406,677

(22) PCT Filed: Jun. 14, 2013
(86) PCT No.: PCT/US13/45843

§ 371 Date: Dec. 9, 2014

Related U.S. Application Data
(60) Provisional application No. 61/660,112, filed on Jun. 15, 2012.

(51) Int. Cl. C12N 5/0797; A61K 38/18; A61K 35/30; C12N 5/077; C12N 5/079; A61K 35/12; A61K 35/32

(52) U.S. Cl. CPC C12N 5/0623; A61K 35/12; A61K 38/1875; A61K 35/32; C12N 5/0653; C12N 5/0622; A61K 35/30

(57) ABSTRACT

The present invention provides an isolated population of perineurium derived adult stem cells and cells derived thereof. The cells of the invention are obtained from the perineurium of peripheral nerves and demonstrate the ability to expand and differentiate in response to BMP2. The invention also provides methods of using the cells of the invention, for example in methods to promote neuroregeneration and bone formation.

### CROSS-REFERENCE TO RELATED APPLICATION

This application claims priority to U.S. Provisional Application Ser. No. 61/660,112, filed Jun. 15, 2012, the content of which is incorporated by reference herein in its entirety.

### STATEMENT REGARDING FEDERALLY SPONSORED RESEARCH OR DEVELOPMENT

This invention was made with government support under HL092332-08, awarded by the National Institutes of Health (NIH) and PR110222 from the U.S. Department of the Army (DOD). The government has certain rights in the invention.

### BACKGROUND OF THE INVENTION

Although depots of brown adipose tissue (BAT) play a critical role in adaptive thermogenesis (Cannon and Nedergaard, 2004, Physiol Rev 84:277-359; Lowell and Spiegelman, 2000, Nature 404:652-660; Bartness et al., 2010, Int J Obes (Lond) 34(Suppl 1):536-42), BAT-like cells (Olmsted-Davis et al., 2007, Am J Pathol 170:620-632), is likely to have other functions. BAT is profoundly involved in triglyceride homeostasis (Bartell et al., 2011, Nat Med 17:200-205) and controls microenvionmental oxygen tension enabling cartilage formation during endochondral ossification (Olmsted-Davis et al., 2007, Am J Pathol 170:620-632). BAT-like cells can also secrete VEGF-D (Dilling et al., 2010, J Bone Miner Res 25:1147-1156), which has roles in vasculogenesis (Song et al., 2007, Biochem Biophys Res Commun 357:924-930), lymphangiogenesis (Kopfstein et al., 2007, Am J Pathol 170:1348-1361), and neuronal arborization (Mauceri et al., 2011, Neuron 71:117-130). Further, defects in BAT biogenesis are likely involved in the pathogenesis of Huntington's disease (Kim et al., 2010, Hum Mol Genet 19:3919-3935; Weydt et al., 2006, Cell Metab 4:349-362), suggesting a key role in the maintenance of neuronal tissues.

Generation of BAT as well as its thermogenic functions are controlled by activation of the sympathetic nervous system (SNS) (Cannon and Nedergaard, 2004, Physiol Rev 84:277-359; Lowell and Spiegelman, 2000, Nature 404:652-660; Klingenspor, 2003, Exp Physiol 88:141-148). Sympathetic nerves can be activated through release of serotonin either locally during neurogenic inflammation (Wilhelm et al., 2005, Eur J Neurosci 22:2238-2248) or systemically through the hypothalamus (Duey and Karsenty, 2010, J Cell Biol 191:7-13; Berger et al., 2009, Annu Rev Med 60:355-366). Binding of serotonin to the 5-HT receptor, leads to release of noradrenaline, which in turn stimulates β-adrenergic receptor (ADRB) signaling pathways involved in the induction of BAT (Collins et al., 2010, Int J Obes (Lond) 34(Suppl 1):S28-33). However, the nature of the progenitors that are stimulated to undergo brown adipogenesis is unclear. Many studies have identified genes thought to be involved in this process including PRDM16 (Seale et al., 2011, J Clin Invest 121:96-105), PPARa (Hondares et al., 2011, J Biol Chem 286(50):43112-22), PGC1α (Puigserver et al., 1998, Cell 92:829-839), Dio2 (de Jesus et al., 2001, J Clin Invest 108:1379-1385), and FoxC2 that lead to the activation of the uncoupling protein 1 (UCP1) gene. UCP1 is a signature protein exclusive to brown adipocytes and the main effector of thermogenesis, which can uncouple oxidative phosphorylation to dissipate energy as heat (Klingenspor, 2003, Exp Physiol 88:141-148; Nedergaard et al., 2001, Biochim Biophys Acta 1504:82-106). However, its role in lowering oxygen tension in the microenvironment (Olmsted-Davis et al., 2007, Am J Pathol 170:620-632) in some instances, may be even more important than its ability to generate heat, since unlike ATP synthase, it is the only enzyme with the throughput necessary to actually accomplish this task.

BAT biogenesis also depends on other key neuronal pathways. Mice lacking a key neuronal protein, Dock 7, no longer produce BAT (Sviderskaya et al., 1998, Genetics 148:381-390; Blasius et al., 2009, Proc Natl Acad Sci USA 106:2706-2711). Dock 7 regulates neuronal polarity and without it axonal growth stops (Watabe-Uchida et al., 2006, Neuron 51:727-739; Pinheiro et al., 2006, Neuron 51:674-676). Dock 7 is phosphorylated by ErbB2 (Yamauchi et al., 2008, J Cell Biol 181:351-365), a protein that is an absolute requisite for the formation of the peripheral nervous system (Lee et al., 1995, Nature 378:394-398). Finally, BAT is highly innervated by both the SNS and sensory nerves (Bartness et al., 2010, Int J Obes (Lond) 34(Suppl 1):S36-42) and ablation of sensory nerves causes transient regression of BAT (Cui et al., 1990, Am J Physiol 259:R324-332).

In addition, β3-agonists increases BAT in mice, dogs, primates, and adult humans (Harper et al., 2008, Annu Rev Nutr 28:13-33). Enhanced noradrenaline release, due to rare tumors of the adrenal glands (pheochromocytomas), also develop more abundant BAT (Lowell and Spiegelman, 2000, Nature 404:652-660; Garruti and Ricquier, 1992, Int J Obes Relat Metab Disord 16:383-390). Although previously not thought to be present in humans, recent studies have contradicted this showing that BAT in humans can be enhanced through exposure to the cold (Nedergaard et al., 2007, Am J Physiol Endocrinol Metab 293:E444-452; van Marken Lichtenbelt et al., 2009, N Engl J Med 360:1500-1508; Virtanen et al., 2009, N Engl J Med 360:1518-1525). However, the nature of the progenitors responding to the SNS signaling remains unclear. Studies examining the origins of BAT have suggested an embryonic Myf5 expressing precursor cell, which can give rise to either skeletal muscle or brown adipocytes, with the transcription factor PRDM16 determining whether skeletal myoblasts or BAT is produced (Seale et al., 2008, Nature 454:961-967; Enerback, 2009, N Engl J Med 360:2021-2023). These studies also show that BAT induced by β3-agonists in white adipose tissue (WAT) do not arise from the same progenitor as interscapular BAT, indicating diversity in the source of BAT. Although WAT progenitors have been shown to reside in WAT vasculature (Tang et al., 2008, Science 322:583-586), brown adipocyte progenitors have not been similarly characterized.

Many reports note that BAT can arise in WAT (Cinti, 2009, Am J Physiol Endocrinol Metab 297(5):E977-86), and the physiological significance of this process was recently underscored by noting the secretion of irisin by muscle to induce UCP1-mediated thermogenesis by WAT (Bostrom et al., 2012, Nature 2012 481:463-468).

It was previously demonstrated that delivery of BMP2 in skeletal muscle has the ability to generate and expand BAT-like cells (Olmsted-Davis et al., 2007, Am J Pathol 170:620-632). It was also found (Salisbury et al., 2011, J Cell Biochem 112(10):2748-58; Kan et al., 2011, J Cell Biochem 112(10):2759-72) that exposure to BMP2 leads to activation and remodeling of sensory nerves, through inflammatory processes, involving degranulation of mast cells. Degranulation of mast cells leads to local release of serotonin, histamine, and other proteases, which has been shown to activate the SNS, leading to the transient appearance of UCP1+BAT.

Despite the progress made in elucidating mechanisms of brown fat adipogenesis, there remains a need to locate and isolate stem cells and precursors which can be develop into BAT and BAT-like cells. The present invention satisfies this unmet need.

### SUMMARY OF THE INVENTION

The invention provides an isolated perineurium derived adult stem cell capable of differentiating into brown adipose tissue.

In one embodiment, the cell expresses β3 adrenergic receptor (ADRB3).

In one embodiment, the cell expands in response to stimulation with BMP-2.

In one embodiment, the invention includes a brown adipose tissue like cell derived from an isolated perineurium derived adult stem cell.

In one embodiment, the brown adipose tissue like cell expresses UCP-1.

In one embodiment, the invention includes an astrocyte like cell derived from an isolated perineurium derived adult stem cell.

In one embodiment, the astrocyte like cell expresses reelin.

In one embodiment, the isolated perineurium derived adult stem cell is pluripotent.

In one embodiment, the isolated perineurium derived adult stem cell retains the ability to differentiate into a germ layer selected from the group consisting of mesoderm, ectoderm, endoderm, and any combination thereof.

In one embodiment, the perineurium derived adult stem cell is isolated from the perineurium of a peripheral nerve.

The invention also provides a method of generating an isolated population of perineurium derived adult stem cells. In one embodiment, the method comprises isolating a peripheral nerve from a subject and extracting cells from the perineurium of the peripheral nerve.

In one embodiment, the method further comprises separating the extracted cells by selecting for cells expressing ADRB3.

In one embodiment, the method further comprises culturing the extracted cells.

The invention also provides a method of promoting bone growth. In one embodiment, the method comprises administering a population of perineurium derived adult stem cells to a region in need of bone growth in a subject.

In one embodiment, the method further comprises administering BMP-2 to the region.

In one embodiment, the perineurium derived adult stem cells are present within a biocompatible scaffold.

In one embodiment, the biocompatible scaffold comprises perineurium derived adult stem cells and osteoblasts.

In one embodiment, the biocompatible scaffold comprises perineurium derived adult stem cells and osteoprogenitor cells.

The invention also provides a method of promoting neuroregeneration. In one embodiment, the method comprises administering a population of perineurium derived adult stem cells to a region in need of neuroregeneration in a subject.

In one embodiment, the method further comprises administering BMP-2 to the region.

In one embodiment, the perineurium derived adult stem cells are present within a biocompatible scaffold.

In one embodiment, the biocompatible scaffold comprises perineurium derived adult stem cells and neural progenitor cells.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A and FIG. 1B, depicts the results of experiments analyzing sympathetic activity during BMP2 induced heterotopic ossification. FIG. 1A depicts a schematic representation of the mechanism leading to sympathetic signaling. Degranulation of mast cells upon activation by neurogenic inflammation, which is initiated upon BMP2 stimulation in a mouse model, leads to the release of 5-HT (serotonin) that can further bind to its receptor on adrenergic neurons. Binding on adrenergic neurons perpetuates downstream sympathetic signaling. FIG. 1B depicts a graph illustrating the measurement of noradrenaline levels, which revealed a significant increase in this effector of sympathetic neurons 2 days after BMP2 induction. Plasma was collected at daily intervals from animals receiving either AdBMP2 (BMP2) or Adempty cassette (control) transduced cells. Noradrenaline levels were quantified by an ELISA and statistically significant changes between the groups determined by the Student's t test; n=3 biological replicates per time point. * Denotes statistical significance. Error bars represent ±SEM (standard error of the mean).

FIG. 2 is a set of images demonstrating the presence of replicating ADRB3+ cells in the perineurial region of peripheral nerves after 2 days induction with BMP2. Green, ADRB3; Red, neurofilament; Red; Ki67 in rightmost panels.

FIG. 3, comprising FIG. 3A through FIG. 3C, is a set of graphs depicting the results of experiments illustrating the induction of brown adipocytes after delivery of BMP2 transduced cells. Muscle tissue, which encompasses the site of new bone formation, was isolated at daily intervals after induction with AdBMP2 (BMP2) or Adempty (control) transduced cells. Total RNA was isolated and subjected to qRT-PCR analysis for quantitation of RNA expression of the adrenergic receptors (FIG. 3A and FIG. 3C) and UCP1 (FIG. 3B) using the AA Ct method. Relative gene expression is therefore represented in relation to control tissues from animals injected with Adempty transduced cells. All assays were performed in duplicate, with n=4 biological replicates per time point. Statistically significant changes were determined by the Student's t test. In FIG. 3B, relative UCP1 gene expression at the mRNA level was elevated about 70 fold in BMP2 samples on day 3 related to control samples. *p<0.05; ***p<0.0005. Error bars represent ±SEM (standard error of the mean).

FIG. 4, comprising FIG. 4A through FIG. 4C, depicts the results of experiments illustrating an increase in ADRB3 positive brown adipocytes in the muscle following induction of heterotopic bone formation with cells expressing BMP2. FIG. 4A depicts the results of an experiment where cells were harvested from the muscle tissue and cell surface expression of ADRB3 was determined by flow cytometry. Results are expressed as the percentage of ADRB3 positive cells found within either control muscle tissues (solid), which received no injection of transduced cells, or muscle tissues which received an injection of BMP2 transduced cells (hatched) and harvested at 2 (white), 3 (light gray), and 4 days (dark gray) following injection. For each experiment, 6 muscle samples for each condition were pooled for flow cytometric analysis. Bar graphs show the average of three independent experiments±SEM. Statistically significant changes were determined by the Student's t test (*p<0.05). ADRB3+ cells were significantly elevated on day 3 and 4, but not day 2. A representative histogram of the percentage of ADRB3+ cells on day 4 is shown. FIG. 4B depicts images where ADRB3+ and ADRB3− cell populations were sorted, cytospun, and immunostained for expression of the brown adipocyte marker, UCP1. UCP1 (red) co-localizes with ADRB3 expression (green). Cells were counterstained with DAPI (blue). FIG. 4C depicts representative photomicrographs of muscle tissue isolated 4 days after receiving cells transduced with AdBMP2 (BMP2) and stained for ADRB3 (brown), which co-aligns with staining for UCP1 (brown) on a serial tissue section, adjacent to the section stained with ADRB3. No staining was observed on paraffin sections of muscle tissue taken 4 days after injection of AdEmpty (control) transduced cells (ADRB3 DAB stain shown).

FIG. 5, comprising FIG. 5A through FIG. 5C, depicts the results of experiments illustrating the analysis of ADRB3 expression in the sciatic nerve after induction of bone formation. FIG. 5A depicts a graph illustrating the results of an experiment where cells were harvested from the sciatic nerve tissue and cell surface expression of ADRB3 was determined by flow cytometry. Results are expressed as the percentage of ADRB3 positive cells found within either control sciatic nerves (solid), from tissues which received no injection of transduced cells, or sciatic nerves from tissues which received an injection of BMP2 transduced cells (hatched) and harvested at 2 (white) or 4 days (dark gray) following injection. For each experiment, 6 sciatic nerve samples for each condition were pooled for flow cytometric analysis. Bar graphs show the average of three independent experiments±SEM test (**p<0.005 versus control). Note the increase in ADRB3+ cells from tissues undergoing HO on day 2, and the decrease on day 4. FIG. 5B depicts representative photomicrographs of sciatic nerve tissue isolated on day 2, stained with ADRB3 antibodies (green, top panel), and counterstained with DAPI. A serial tissue section, adjacent to the section stained with ADRB3, was stained with antibodies to UCP1. Hematoxylin and eosin stained sections from the same tissue are shown. Arrow indicates the perineurium. FIG. 5C depicts representative photomicrographs of the same sciatic nerve tissue samples shown in FIG. 5B dual-stained for both ADRB3 (green) and Ki67 (red). A merger of these stains shows co-localization of the replication marker Ki67 in some ADRB3 positive cells (top panel). Sciatic nerve tissues from control animals show minimal staining for Ki67 (red, bottom panel).

FIG. 6, comprised of FIG. 6A through FIG. 6C, is a set of images depicting the detailed analysis of ADRB3 and Ki67 expression 3 days after BMP2 induction. Green, ADRB3; Red, Ki67

FIG. 7 depicts the results of experiments illustrating the analysis of HNK expression 3 days after BMP2 induction. Top panel, staining pattern 3 days after BMP2 induction. Bottom panel: FACS analysis for HNK 3 days after injection of either cells transduced with empty vector or BMP2. Total cells from muscle in the area surrounding the site of injection were isolated and subjected to FACS analysis for HNK1.

FIG. 8, comprising FIG. 8A through FIG. 8C, depicts the results of experiments demonstrating the suppression of brown fat induction in tissues undergoing HO in the presence of cromolyn. FIG. 8A and FIG. 8B depict graphs illustrating the results of experiments where total RNA was isolated after the induction of HO by injection of BMP2 transduced cells in animals pretreated with cromolyn (BMP2+cromolyn) or left untreated (BMP2). Relative gene expression in animals treated with BMP2 and cromolyn was expressed in relation to animals treated with BMP2 alone using the AA Ct method. ADRB3 (FIG. 8A) and UCP1 (FIG. 8B) relative gene expression was suppressed in the cromolyn treated animals, with a 39 fold suppression of UCP1 on day 3. Each assay was performed in duplicate with n=4 biological replicates per time point. * Denotes a statistically significant change as determined by a Student's t test (*p<0.05). FIG. 8C depicts a set of photomicrographs of tissues isolated 2 days after induction of HO by injection of BMP2 transduced cells in animals pretreated with cromolyn (BMP2+cromolyn) or left untreated (BMP2) Immunohistochemical staining for UCP1 expression (green) within the nerves, identified by neurofilament staining (pink), of the isolated hind limb tissues. Tissues were counterstained with DAPI (blue).

FIG. 9 is a set of images illustrating that transient brown fat expresses reelin. Upper panel: Reelin, UCP1, and ADRB3 expression were assessed three days after BMP2 induction. Lower panel: ADRB3+ cells were isolated by FACS three days after BMP2 induction. These cells were centrifuged onto microscope slides and labeled with either ADRB3, reelin, or UCP1.

FIG. 10, comprising FIG. 10A and FIG. 10B, is a set of graphs illustrating that the expression of PRDM16 (FIG. 10A) and PPARγ (FIG. 10B), two genes known to be altered in traditional BAT biogenesis, is not significantly increased after BMP-2 stimulation in the present cells.

FIG. 11, comprising FIG. 11A and FIG. 11B, is a set of images depicting the results of experiments illustrating the staining of nerve sections with ADRB3 and other markers. FIG. 11A depicts the staining of a nerve section with ADRB3 and HNK, a neuronal migratory marker. FIG. 11B depicts the staining of a nerve section with ADRB3 and B3GAT2, a protein involved in the synthesis of HNK, 3 days post BMP2 stimulation.

FIG. 12 is an image depicting cells isolated from human peripheral nerves. Human peripheral nerves were cut into 3 mm pieces and placed directly into tissue culture wells, in DMEM supplemented with 10% fetal bovine serum. These cells were allowed to expand and migrate from the nerve for one week, and then the piece of nerve was transferred to a new well. The remaining cells were allowed to expand, and were transferred to dishes containing cover slips for immunostaining, or expanded and frozen in 10% DMSO to make cell stocks. This was done for four weeks, until the nerve pieces were no longer remaining intact. Cells from each nerve passage were labeled and retained separately. All stains within this figure were from first passage cells. Second, third and fourth passage cells were immunostainned but either were mixed populations or lacked the UCP1⁺ cells.

FIG. 13 is a set of photomicrographs of human tibial nerve. Serial sections from the nerve were immunostainned for ADRB3 and UCP1, to identify positive cells. Note in the hematoxylin and eosin stained images, fasicles that lack a distinct perineurium appear to have significant ADRB3 expression and UCP1, suggesting that in those sections the progenitors have formed brown adipose rather than the differentiated perineurial fibroblast.

### DETAILED DESCRIPTION

The present invention provides isolated perineurium derived adult stem cells and downstream cells derived thereof. In one embodiment, the perineurium derived adult stem cells are stimulated with BMP2, noradrenaline, or a combination thereof to induce the expansion, differentiation, and migration of the cells. In one embodiment, the cells of the invention are brown adipose tissue like cells (BALCs), expressing the traditional BAT marker, UCP-1. However, in one embodiment, the BALCs do not demonstrate changes in the expression of genes such as PRDM16, PPARγ, PPARα, PPARΔ that are traditionally associated with BAT biogenesis. In one embodiment, the cells of the invention are astrocyte-like cells, expressing for example reelin. In one embodiment, the perineurium derived adult stem cells are characterized by the presence of β3 adrenergic receptor (ADRB3). Thus, in one embodiment, the perineurium derived stem cells are ADRB3+.

The present invention also provides methods of using isolated perineurium derived adult stem cells and cells derived thereof. In one embodiment, the cells of the invention have potential to differentiate into a desired cell type. In another embodiment, the cells of the invention can support bone growth and bone repair. Thus, in one embodiment, the cells of the invention can be used in cell therapy and tissue engineering applications to promote bone growth in vivo, ex vivo, or in vitro. In one embodiment, the cells of the invention can support neuroregeneration, including for example neuronal regeneration, axonal regeneration, peripheral nerve regeneration, and the like. Thus, in one embodiment, the cells of the invention can be used in cell therapy and tissue engineering applications to treat spinal cord injury, peripheral nerve injury, neuropathic pain, and neurodegenerative disorders. Other methods of the invention include the use of the cells to treat obesity, diabetes, cancer, and vascular calcification such as atherosclerosis or calcific aortic valve disease.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

“Astrocyte-like cell” is used herein to refer to a cell that exhibits a phenotype similar to that of an astrocyte and which expresses the astrocyte-specific marker, such as, but not limited to, GFAP.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

As used herein “conditioned media” defines a medium in which a specific cell or population of cells have been cultured in, and then removed. While the cells were cultured in said medium, they secrete cellular factors that include, but are not limited to hormones, cytokines, extracellular matrix (ECM), proteins, vesicles, antibodies, and granules. The medium plus the cellular factors is the conditioned medium.

The term “dedifferentiation”, as used herein, refers to the return of a cell to a less specialized state. After dedifferentiation, such a cell will have the capacity to differentiate into more or different cell types than was possible prior to re-programming. The process of reverse differentiation (i.e., de-differentiation) is likely more complicated than differentiation and requires “re-programming” the cell to become more primitive.

“Differentiated” is used herein to refer to a cell that has achieved a terminal state of maturation such that the cell has developed fully and demonstrates biological specialization and/or adaptation to a specific environment and/or function. Typically, a differentiated cell is characterized by expression of genes that encode differentiation associated proteins in that cell. When a cell is said to be “differentiating,” as that term is used herein, the cell is in the process of being differentiated.

“Differentiation medium” is used herein to refer to a cell growth medium comprising an additive or a lack of an additive such that a stem cell, adipose derived adult stromal cell or other such progenitor cell, that is not fully differentiated when incubated in the medium, develops into a cell with some or all of the characteristics of a differentiated cell.

As used herein “development controllers” is intended the following non-limiting controllers including, but are not limited to embryonic development markers, nervous system development markers, central nervous system development markers, muscle development markers, skeletal development markers, cartilage development markers, ovarian follicle development, and the like. Angiogenic Growth Factors include but are not limited to ARTS-1, ECGF1, EREG, FGF1, FGF2, FGF6, FIGF, IL18, JAG1, PGF, TNNT1, VEGFA, VEGFC, and the like. Cell Differentiation markers include but are not limited to ARTS-1, BMP1, BMP2, BMP3, BMP4, BMP5, BMP6, BMP7, BMP8B, CSF1, CSPGS, ECGF1, EREG, FGF1, FGF2, FGF22, FGF23, FGF6, FGF9, FIGF, IL10, IL11, IL12B, IL2, IL4, INHA, INHBA, INHBB, JAG1, JAG2, LTBP4, MDK, NRG1, OSGIN1 (OKL38), PGF, SLCO1A2, SPP1, TDGF1, TNNT1, VEGFC, and the like. Embryonic Development markers include but are not limited to BMP10, NRG1, NRG2, NRG3, TDGF1, and the like. Nervous System Development markers include but are not limited to BDNF, CSPGS, CXCL1, FGF11, FGF13, FGF14, FGF17, FGF19, FGF2, FGF5, GDF11, GDNF, GPI, IL3, INHA, INHBA, JAG1, MDK, NDP, NRG1, NRTN, NTF3, PTN, VEGFA, and the like. Central Nervous System Development markers include but are not limited to PDGFC, PSPN, and the like. Muscle Development markers include but are not limited to FGF2, GDF8, HBEGF, IGF1, TNNT1, and the like. Skeletal Development markers include but are not limited to GDF10, GDF11, IGF1, IGF2, INHA, INHBA, and the like. Cartilage Development markers include but are not limited to BMP2, BMP3, BMP4, BMP5, BMP6, BMP7, BMP8B. Ovarian Follicle Development markers include but are not limited to INHA, INHBA, INHBB, and the like. Others markers include but are not limited to AMH, CECR1, CSF2, CSF3, DKK1, FGF7, LEFTY1, LEFTY2, LIF, LTBP4, NGFB, NODAL, TGFB1, THPO, and the like.

The term “derived from” is used herein to mean to originate from a specified source.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

“Expandability” is used herein to refer to the capacity of a cell to proliferate, for example, to expand in number or in the case of a cell population to undergo population doublings.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

As used herein “growth factors” is intended the following non-limiting factors including, but not limited to, growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor (EGF), fibroblast growth factor (FGF), nerve growth factor, ciliary neurotrophic factor, platelet derived growth factor (PDGF), transforming growth factor (TGF-beta), hepatocyte growth factor (HGF), and bone morphogenetic protein at concentrations of between picogram/ml to milligram/ml levels.

As used herein, the term “growth medium” is meant to refer to a culture medium that promotes growth of cells. A growth medium will generally contain animal serum. In some instances, the growth medium may not contain animal serum.

An “isolated cell” refers to a cell which has been separated from other components and/or cells which naturally accompany the isolated cell in a tissue or mammal.

As used herein, the term “multipotential” or “multipotentiality” is meant to refer to the capability of a stem cell to differentiate into more than one type of cell.

“Oligodendrocyte-like cell” is used herein to refer to a cell that exhibits a phenotype similar to that of an oligodendrocyte and which expresses the oligodendrocyte-specific marker, such as, but not limited to, 0-4.

As used herein, a “pluripotent cell” defines a less differentiated cell that can give rise to at least two distinct (genotypically and/or phenotypically) further differentiated progeny cells.

The terms “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew itself or to produce progeny cells which will differentiate into the desired cell type. Unlike pluripotent stem cells, lineage-committed progenitor cells are generally considered to be incapable of giving rise to numerous cell types that phenotypically differ from each other. Instead, progenitor cells give rise to one or possibly two lineage-committed cell types.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cell, and the like.

“Progression of or through the cell cycle” is used herein to refer to the process by which a cell prepares for and/or enters mitosis and/or meiosis. Progression through the cell cycle includes progression through the G1 phase, the S phase, the G2 phase, and the M-phase.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, “tissue engineering” refers to the process of generating tissues ex vivo for use in tissue replacement or reconstruction. Tissue engineering is an example of “regenerative medicine,” which encompasses approaches to the repair or replacement of tissues and organs by incorporation of cells, gene or other biological building blocks, along with bioengineered materials and technologies.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to isolated perineurium derived adult stem cells and cells derived therefrom and methods of using such cells in any application including but is not limited to therapeutic and tissue engineering. In one embodiment, the cells of the invention reside the perineurium of peripheral nerves and can be isolated therefrom. The invention is based upon the discovery that cells expressing ADRB3 in the perineurium expand, differentiate, and migrate in response to stimulation with BMP2. The invention further provides methods of isolating, culturing, expanding, and using perineurium derived adult stems.

### Compositions

The present invention provides an isolated population of perineurium derived adult stem cells, and cells derived thereof. Cells derived from the perineurium derived adult stem cells include, but are not limited to cells that differentiate from, dedifferentiate from, propagate from, and are downstream progeny of the perineurium derived adult stem cells.

The perineurium derived adult stem cells of the invention can differentiate into cells that give rise to more than one type of germ layer, e.g. mesoderm, endoderm, or ectoderm, and a combination thereof.

In another embodiment, the perineurium derived adult stem cells can differentiate into two or more distinct lineages from different germ layers (such as endodermal and mesodermal), for example hepatocytes and adipocytes.

The perineurium derived adult stem cells of the invention can differentiate into cells of two or more lineages, for example adipogenic, chondrogenic, cardiogenic, dermatogenic, hematopoetic, hemangiogenic, myogenic, nephrogenic, neurogenic, neuralgiagenic, urogenitogenic, osteogenic, pericardiogenic, peritoneogenic, pleurogenic, splanchogenic, and stromal developmental phenotypes. While such cells can retain two or more of these different lineages (or developmental phenotypes), preferably, such perineurium derived adult stem cells can differentiate into three or more different lineages. The most preferred perineurium derived adult stem cells can differentiate into four or more lineages.

The perineurium derived adult stem cells of the invention may differentiate into mesodermal tissues, such as mature adipose tissue, bone, various tissues of the heart (e.g., pericardium, epicardium, epimyocardium, myocardium, pericardium, valve tissue, etc.), dermal connective tissue, hemangial tissues (e.g., corpuscles, endocardium, vascular epithelium, etc.), hematopoetic tissue, muscle tissues (including skeletal muscles, cardiac muscles, smooth muscles, etc.), urogenital tissues (e.g., kidney, pronephros, meta- and meso-nephric ducts, metanephric diverticulum, ureters, renal pelvis, collecting tubules, epithelium of the female reproductive structures (particularly the oviducts, uterus, and vagina), mesodermnal glandular tissues (e.g., adrenal cortex tissues), and stromal tissues (e.g., bone marrow). Of course, inasmuch as the perineurium derived stem cell can retain potential to develop into a mature cell, it also can realize its developmental phenotypic potential by differentiating into an appropriate precursor cell (e.g., a preadipocyte, a premyocyte, a preosteocyte, etc.).

In another embodiment, the perineurium derived adult stem cells may differentiate into ectodermal tissues, such as neurogenic tissue, and neurogliagenic tissue.

In another embodiment, the perineurium derived adult stem cells may differentiate into endodermal tissues, such as pleurogenic tissue, and splanchnogenic tissue, and hepatogenic tissue, and pancreogenic tissue.

In yet another embodiment, perineurium derived adult stem cells may dedifferentiate into developmentally immature cell types. Examples of perineurium derived adult stem cells dedifferentiating into an immature cell type, include embryonic cells and fetal cells or embryonic-like and fetal-like cells.

In another embodiment, the inventive perineurium derived adult stem cells can give rise to one or more cell lineages from one or more germ layers such as neurogenic cells (of ectodermal origin) and myogenic cells (of mesodermal origin).

The perineurium derived adult stem cells of the invention are ADRB3+ and can be found within the perineurium of peripheral nerves. Upon stimulation with BMP2, the perineurium derived adult stem cells expand, differentiate, and migrate towards BMP2. In one embodiment, the stimulated perineurium derived adult stem cells differentiate into a BAT like cell. That is, in some instances the stimulated cells of the invention have features indicative of BAT. For example, the stimulated cells of the invention express UCP-1, a presumptive BAT marker. In one embodiment, the stimulated cells of the invention differentiate into an astrocyte like cell. That is, in some instances the stimulated cells of the invention have features indicative of an astrocyte. For example, the stimulated cells of the invention express reelin, a protein typically found in astrocytes.

In one embodiment, the perineurium derived adult stem cells of the invention differentiate into support cells. In one embodiment, the support cells aid in establishing a proper environment for tissue repair or tissue regeneration. For example, the supports cells produce microenvironmental cues such as hypoxia and neovascularization. Such cues are beneficial in applications such as bone growth, nerve outgrowth, and cartilage development.

### Methods of Obtaining and Culturing Cells of the Invention

The perineurium derived adult stem cells of the invention are isolated from the perineurium of peripheral nerves. The peripheral nervous system (PNS) comprises the nerves and ganglia outside of the brain and spinal cord. The PNS can be divided into several different sections including the sensory nervous system, the motor nervous system, the somatic nervous system, the autonomic nervous system, sympathetic neurons, and parasympathetic neurons. The nerves of the PNS carry information to and from the central nervous system (CNS). The PNS nerve fibers are wrapped in a sheath known as the endoneurium. These wrapped fibers are bundled together to form fascicles, where each fascicle is surrounded by the perineurium. Several wrapped fascicles are then bundled together with blood vessels and fatty tissue within the epineurium. The present invention is partly based on the discovery of stem cells existing within the perineurium of PNS nerves. However, in some embodiments, the cells of the invention are found and isolated from the endoneurium or the epineurium.

The perineurium is generally comprised of several concentric layers and is composed of flattened cells, basement membrane and collagen fibers (Pina-Oviedo, 2008, Adv. Anat. Pathol., 15(3): 147-64). The perineurium functions to prevent external stretching and to form a nerve-blood barrier to the nerve fibers. In some instances, the perineurium is a smooth tubular layer that can be separated from the enclosed nerve fibers.

In the isolation of the cells of the invention, the perineurium can be obtained from any animal by any suitable method. A first step in any such method requires the isolation of the perineurium from the source animal. The animal can be alive or dead, so long as cells within the perineurium are viable. Typically, human perineurium is obtained from a living donor, using well-recognized surgical protocols. The perineurium derived adult stem cells of the invention are present in the initially excised or extracted perineurium, regardless of the method by which the perineurium is obtained. The perineurium may be obtained from any peripheral nerve within the PNS. Non-limiting examples of peripheral nerves in the human from which the perineurium may be obtained includes sciatic nerve, pudendal nerve, femoral nerve, subcostal nerve, intercostal nerves, musculocutaneous nerve, radial nerve, median nerve, iliohypogastric nerve, genitofemoral nerve, obturator nerve, ulnar nerve, common peroneal nerve, deep peroneal nerve, superifical peroneal nerve, nerves of the brachial plexus, nerves of the lumbar plexus, nerves of the sciatic plexus, and nerves of the cervical plexus. In another embodiment, the perineurium may be obtained from the PNS of non-human animals.

In one embodiment, a peripheral nerve or section of peripheral nerve is removed from the animal. In one embodiment, the perineurium is separated from the rest of the excised nerve. Separation of the perineurium from the nerve can be achieved by any method known in the art. For example, in one embodiment, the epineurium is cut away from the rest of the nerve, exposing the perineurium wrapped fascicles. In one embodiment, the perineurium is separated from the nerve fibers by surgical or enzymatic means. However obtained, the perineurium is processed to separate the perineurium derived adult stem cells of the invention from the remainder of the perineurium. In one embodiment, the perineurium is washed with a physiologically-compatible solution, such as phosphate buffer saline (PBS). The washing step consists of rinsing the perineurium tissue with PBS, agitating the tissue, and allowing the tissue to settle. In one embodiment, the perineurium is dissociated. The dissociation can occur by enzyme degradation and neutralization. Alternatively, or in conjunction with such enzymatic treatment, other dissociation methods can be used such as mechanical agitation, sonic energy, or thermal energy.

In some instances, it may be desirable to further process the dissociated tissue. For example, the dissociated perineurium can be filtered to isolate cells from other connective tissue. The extracted cells can be concentrated into a pellet. One method to concentrate the cells includes centrifugation, wherein the sample is centrifuged and the pellet retained. The pellet includes the perineurium derived adult stem cells of the invention.

In one embodiment, the cells are resuspended and can be washed (e.g. in PBS). Cells can be centrifuged and resuspended successive times to achieve a greater purity. In one embodiment, the cells extracted from the perineurium may be a heterogeneous population of cell which includes the perineurium derived adult stem cells of the invention. The perineurium derived adult stem cells may be separated from other cells by methods that include, but are not limited to, cell sorting, size fractionation, granularity, density, molecularity, morphologically, and immunohistologically. In one embodiment, perineurium derived adult stem cells of the invention are separated from other cells by assaying the length of the telomere, as stem cells tend to have longer telomeres compared to differentiated cells. In another embodiment, perineurium derived adult stem cells of the invention are separated from other cells by assaying telomeric activity, as telomeric activity can serve as a stem-cell specific marker. In another embodiment, perineurium derived adult stem cells of the invention are separated from other cells immunohistochemically, for example, by panning, using magnetic beads, or affinity chromatography. For example, in one embodiment, the perineurium derived adult stem cells can be separated through positive selection of ADRB3 located on the surface of the perineurium derived adult stem cells. Separation of cells may be carried out through positive selection, negative selection, or depletion. Such methods are well known in the art.

The perineurium derived adult stem cells can be cultured and, if desired, assayed for number and viability, to assess the yield. In one embodiment, the stem cells are cultured without differentiation using standard cell culture media (e.g., DMEM, typically supplemented with 5-15% (e.g., 10%) serum (e.g., fetal bovine serum, horse serum, etc.). In one embodiment, the stem cells are passaged at least five times in such medium without differentiating, while still retaining their developmental phenotype. In one embodiment, the stem cells are passaged at least 10 times (e.g., at least 15 times or even at least 20 times) while retaining potency.

The perineurium derived adult stem cells can be separated by phenotypic identification, to identify those cells that have two or more of the aforementioned developmental lineages. In one embodiment, all cells extracted from the perineurium are cultured. To phenotypically separate the perineurium derived adult stem cells from the other cells of the perineurium, the cells are plated at a desired density, such as between about 100 cells/cm² to about 100,000 cells/cm² (such as about 500 cells/cm² to about 50,000 cells/cm², or, more particularly, between about 1,000 cells/cm² to about 20,000 cells/cm²).

In one embodiment the extracted cells of the perineurium is plated at a lower density (e.g., about 300 cells/cm²) to facilitate the clonal isolation of the perineurium derived adult stem cells. For example, after a few days, perineurium derived adult stem cells plated at such densities will proliferate (expand) into a clonal population of perineurium derived adult stem cells.

Such perineurium derived adult stem cells can be used to clone and expand a multipotent perineurium derived adult stem cell into clonal populations, using a suitable method for cloning cell populations. The cloning and expanding methods include cultures of cells, or small aggregates of cells, physically picking and seeding into a separate plate (such as the well of a multi-well plate). Alternatively, the stem cells can be subcloned onto a multi-well plate at a statistical ratio for facilitating placing a single cell into each well (e.g., from about 0.1 to about 1 cell/well or even about 0.25 to about 0.5 cells/well, such as 0.5 cells/well). The perineurium derived adult stem cells can be cloned by plating them at low density (e.g., in a petri-dish or other suitable substrate) and isolating them from other cells using devices such as a cloning rings. Alternatively, where an irradiation source is available, clones can be obtained by permitting the cells to grow into a monolayer and then shielding one and irradiating the rest of cells within the monolayer. The surviving cell then will grow into a clonal population. Production of a clonal population can be expanded in any suitable culture medium, for example, an exemplary culture condition for cloning stem cells (such as the inventive stem cells or other stem cells) is about ⅔ F12 medium+20% serum (e.g. fetal bovine serum) and about ⅓ standard medium that has been conditioned with stromal cells, the relative proportions being determined volumetrically).

In any event, whether clonal or not, the isolated perineurium derived adult stem cells can be cultured in a specific inducing medium to induce the perineurium derived adult stem cells to differentiate and express its multipotency. The perineurium derived adult stem cells give rise to cells of mesodermal, ectodermal and endodermal lineage, and combinations thereof. Thus, perineurium derived adult stem cells can be treated to differentiate into a variety of cell types.

In another embodiment, the perineurium derived adult stem cells are cultured in a defined medium for inducing adipogenic differentiation. Examples of specific media that induce the cells of the invention to take on a adipogenic phenotype include, but are not limited to media containing a glucocorticoid (e.g., dexamethasone, hydrocortisone, cortisone, etc.), insulin, a compound which elevates intracellular levels of cAMP (e.g., dibutyryl-cAMP, 8-CPT-cAMP (8-(4)chlorophenylthio)-adenosine 3′,5′ cyclic monophosphate; 8-bromo-cAMP; dioctanoyl-cAMP, forskolin etc.), and/or a compound which inhibits degradation of cAMP (e.g., a phosphodiesterase inhibitor such as isobutyl methyl xanthine (IBMX), methyl isobutylxanthine, theophylline, caffeine, indomethacin, and the like), and serum. Thus, exposure of the perineurium derived adult stem cells to between about 1 μM and about 10 μM insulin in combination with about 10⁻⁹ M to about 10⁻⁶ M to (e.g., about 1 μM) dexamethasone can induce adipogenic differentiation. Such a medium also can include other agents, such as indomethacin (e.g., about 100 μM to about 200 μM), if desired, and preferably the medium is serum-free.

In another embodiment, perineurium derived adult stem cells cultured in DMEM, 10% FBS, 1 μM dexamthasone, 10 μM insulin, 200 μM indomethacin, 1% antibiotic/antimicotic(ABAM), 0.5 mM IBMX, take on an adipogenic phenotype.

Culturing media that can induce osteogenic differentiation of the perineurium derived adult stem cells include, but are not limited to, about 10⁻⁷ M and about 10⁻⁹ M dexamethasone in combination with about 10 μM to about 50 μM ascorbate-2-phosphate and between about 10 nM and about 50 nM β-glycerophosphate. The medium also can include serum (e.g., bovine serum, horse serum, etc.).

In another embodiment, perineurium derived adult stem cells cultured in DMEM, 10% FBS, 5% horse serum, 50 μM hydrocortisone, 10⁻⁷M dexamethosone, 50 μM ascorbate-2-phosphate, 1% ABAM, take on an osteogenic phenotype.

Culturing medium that can induce myogenic differentiation of the perineurium derived adult stem cells of the invention include, but is not limited to, exposing the cells to between about 10 μM and about 100 μM hydrocortisone, preferably in a serum-rich medium (e.g., containing between about 10% and about 20% serum (either bovine, horse, or a mixture thereof)). Other glucocorticoids that can be used include, but are not limited to, dexamethasone. Alternatively, 5′-azacytidine can be used instead of a glucocorticoid.

In another embodiment, perineurium derived adult stem cells cultured in DMEM, 10% FBS, 10⁻⁷M dexamethosone, 50 μM ascorbate-2-phosphate, 10 mM beta-glycerophosphate, 1% ABAM, take on an myogenic phenotype.

Culturing medium that can induce chondrogenic differentiation of the perineurium derived adult stem cells of the invention, include but is not limited to, exposing the cells to between about 1 μM to about 10 μM insulin and between about 1 μM to about 10 μM transferrin, between about 1 ng/ml and 10 ng/ml transforming growth factor (TGF) β1, and between about 10 nM and about 50 nM ascorbate-2-phosphate. For chondrogenic differentiation, preferably the cells are cultured in high density (e.g., at about several million cells/ml or using micromass culture techniques), and also in the presence of low amounts of serum (e.g., from about 1% to about 5%).

In another embodiment, perineurium derived adult stem cells cultured in DMEM, 50 μM ascorbate-2-phosphate, 6.25 μg/ml transferin, 10 ng/ml insulin growth factor (IGF-1), 5 ng/ml TGF-beta-1, 5 ng/ml basic fibroblast growth factor (bFGF; used only for one week), assume an chondrogenic phenotype.

In yet another embodiment, perineurium derived adult stem cells are cultured in a neurogenic medium such as DMEM, no serum and 5-10 mM (3-mercaptoethanol and assume an ectodermal lineage.

The perineurium derived adult stem cells also can be induced to dedifferentiate into a developmentally more immature phenotype (e.g., a fetal or embryonic phenotype). Such an induction is achieved upon exposure of the perineurium derived adult stem cells to conditions that mimic those within fetuses and embryos. For example, the inventive perineurium derived adult stem cells, can be co-cultured with cells isolated from fetuses or embryos, or in the presence of fetal serum.

The perineurium derived adult stem cells of the invention can be induced to differentiate into a mesodermal, ectodermal, or an endodermal lineage by co-culturing the cells of the invention with mature cells from the respective germ layer, or precursors thereof.

In an embodiment, induction of the perineurium derived adult stem cells into specific cell types by co-culturing with differentiated mature cells includes, but is not limited to, myogenic differentiation induced by co-culturing the perineurium derived adult stem cells with myocytes or myocyte precursors. Induction of the perineurium derived adult stem cells into a neural lineage by co-culturing with neurons or neuronal precursors, and induction of the perineurium derived adult stem cells into an endodermal lineage, may occur by co-culturing with mature or precursor pancreatic cells or mature hepatocytes or their respective precursors.

Alternatively, the perineurium derived adult stem cells are cultured in a conditioned medium and induced to differentiate into a specific phenotype. Conditioned medium is medium which was cultured with a mature cell that provides cellular factors to the medium such as cytokines, growth factors, hormones, and extracellular matrix. For example, a medium that has been exposed to mature myoctytes is used to culture and induce perineurium derived adult stem cells to differentiate into a myogenic lineage. Other examples of conditioned media inducing specific differentiation include, but are not limited to, culturing in a medium conditioned by exposure to heart valve cells to induce differentiation into heart valve tissue. In addition, perineurium derived adult stem cells are cultured in a medium conditioned by neurons to induce a neuronal lineage, or conditioned by hepatocytes to induce an endodermal lineage.

For co-culture, it may be desirable for the perineurium derived adult stem cells and the desired other cells to be co-cultured under conditions in which the two cell types are in contact. This can be achieved, for example, by seeding the cells as a heterogeneous population of cells onto a suitable culture substrate. Alternatively, the perineurium derived adult stem cells can first be grown to confluence, which will serve as a substrate for the second desired cells to be cultured within the conditioned medium.

Other methods of inducing differentiation are known in the art and can be employed to induce the perineurium derived adult stem cells to give rise to cells having a mesodermal, ectodermal or endodermal lineage.

After culturing the stem cells of the invention in the differentiating-inducing medium for a suitable time (e.g., several days to a week or more), the perineurium derived adult stem cells can be assayed to determine whether, in fact, they have acquired the desired lineage.

Methods to characterize differentiated cells that develop from the perineurium derived adult stem cells of the invention, include, but are not limited to, histological, morphological, biochemical and immunohistochemical methods, or using cell surface markers, or genetically or molecularly, or by identifying factors secreted by the differentiated cell, and by the inductive qualities of the differentiated perineurium derived adult stem cells.

Molecular markers that characterize mesodermal cell that differentiate from the cells of the invention, include, but are not limited to, MyoD, myosin, alpha-actin, brachyury, xFOG, Xtbx5 FoxF1, XNkx-2.5. Mammalian homologs of the above mentioned markers are preferred.

Molecular markers that characterize ectodermal cell that differentiate from the cells of the invention, include but are not limited to N-CAM, GABA and epidermis specific keratin. Mammalian homologs of the above mentioned markers are preferred.

Molecular markers that characterize endodermal cell that differentiate from the cells of the invention include, but are not limited to, Xhbox8, Endo1, Xhex, Xcad2, Edd, EF1-alpha, HNF3-beta, LFABP, albumin, insulin. Mammalian homologs of the above mentioned markers are preferred.

In an embodiment, molecular characterization of the differentiated perineurium derived adult stem cells is by measurement of telomere length. Undifferentiated stem cells have longer telomeres than differentiated cells; thus the cells can be assayed for the level of telomerase activity. Alternatively, RNA or proteins can be extracted from the perineurium derived adult stem cells and assayed (via Northern hybridization, RT-PCR, Western blot analysis, etc.) for the presence of markers indicative of a specific phenotype.

In an alternative embodiment, differentiation is assessed by assaying the cells immunohistochemically or histologically, using tissue-specific antibodies or stains, respectively. For example, to assess adipogenic differentiation, the differentiated perineurium derived adult stem cells are stained with fat-specific stains (e.g., oil red O, safarin red, sudan black, etc.) or with labeled antibodies or molecular markers that identify adipose-related factors (e.g., PPAR-γ, adipsin, lipoprotein lipase, etc.).

In another embodiment, osteogenesis can be assessed by staining the differentiated perineurium derived adult stem cells with bone-specific stains (e.g., alkaline phosphatase, von Kossa, etc.) or with labeled antibodies or molecular markers that identify bone-specific markers (e.g., osteocalcin, osteonectin, osteopontin, type I collagen, bone morphogenic proteins, cbfa, etc.).

Myogensis can be assessed by identifying classical morphologic changes (e.g., polynucleated cells, syncitia formation, etc.), or assessed biochemically for the presence of muscle-specific factors (e.g., myo D, myosin heavy chain, etc.).

Chondrogenesis can be determined by staining the cells using cartilage-specific stains (e.g., Alcian blue) or with labeled antibodies or molecular markers that identify cartilage-specific molecules (e.g., sulfated glycosaminoglycans and proteoglycans, keratin, chondroitin, Type II collagen, etc.) in the medium.

Alternative embodiments can employ methods of assessing developmental phenotype, known in the art. For example, the cells can be sorted by size and granularity. The cells can be used as an immunogen to generate monoclonal antibodies (Kohler and Milstein), which can then be used to bind to a given cell type. Correlation of antigenicity can confirm that the perineurium derived adult stem cells has differentiated along a given developmental pathway.

While an perineurium derived adult stem cell can be isolated, preferably it is within a population of cells. The invention provides a defined population of perineurium derived adult stem cells. In an embodiment, the population is heterogeneous. In another embodiment, the population is homogeneous. In another embodiment, a population of perineurium derived adult stem cells can support cells for culturing other cells. For example, cells that can be supported by perineurium derived adult stem cells populations include other types of stem cells, such as neural stem cells (NSC), hematopoetic stem cells (HPC, particularly CD34.sup.+stem cells), embryonic stem cells (ESC) and mixtures thereof), osteoblasts, neurons, chondrocytes, myocytes, and precursors thereof. In other embodiments, the population is substantially homogeneous, consisting essentially of the inventive perineurium derived adult stem cells.

It is described herein, that the perineurium derived adult stem cells of the invention expand, differentiate, and migrate in response to stimulation with BMP2. Further, it is described that this behavior is dependent on sympathetic nervous system signaling, including release of noradrenaline. Therefore, in one embodiment, the isolated perineurium derived adult stem cells of the invention are stimulated with BMP2, noradrenaline, or a combination thereof. In one embodiment, stimulation occurs during culture of isolated perineurium derived adult stem cells. In another embodiment, BMP2, noradrenaline, or a combination thereof is administered to a human or non-human subject prior to isolation of the perineurium. Administration of the BMP and/or noradrenaline to a subject can be performed by any method known in the art. In one embodiment, the perineurium is isolated within a defined time period post stimulation. For example, in one embodiment, the perineurium is isolated about 2 days after stimulation.

### Methods of Use

The perineurium can be used as a source of the perineurium derived adult stem cells of the invention. The dissociated perineurium can be introduced into a subject for tissue regeneration, wound repair or other applications requiring a source of stem cells. In addition, the perineurium can be treated to cause the perineurium derived adult stem cells therein to differentiate into a desired cell type for introduction into a subject. The perineurium derived adult stem cells can also be cultured in vitro to maintain a source of perineurium derived adult stem cells, or can be induced to produce further differentiated perineurium derived adult stem cells that can develop into a desired tissue.

The perineurium derived adult stem cells can be employed for a variety of purposes. The perineurium derived adult stem cells can support the growth and expansion of other cell types. The invention includes a method of conditioning culture medium using the perineurium derived adult stem cells in a suitable medium, and the perineurium derived adult stem cell-conditioned medium produced by such a method. Typically, the medium is used to support the in vitro growth of the perineurium derived adult stem cells, which secrete hormones, cell matrix material, and other factors into the medium. After a suitable period (e.g., one or a few days), the culture medium containing the secreted factors can be separated from the cells and stored for future use. The perineurium derived adult stem cells can be re-used successively to condition medium, as desired. In other applications (e.g., for co-culturing the perineurium derived adult stem cells with other cell types), the cells can remain within the conditioned medium. Thus, the invention provides an perineurium derived adult stem cell-conditioned medium obtained using this method, which either can contain the perineurium derived adult stem cells, or be substantially free of the perineurium derived adult stem cells, as desired.

The perineurium derived adult stem cells-conditioned medium can be used to support the growth and expansion of desired cell types, and the invention provides a method of culturing cells (particularly stem cells) using the conditioned medium. The method involves maintaining a desired cell in the conditioned medium under conditions for the cell to remain viable. The cell can be maintained under any suitable condition for culturing them, such as are known in the art. Desirably, the method permits successive rounds of mitotic division of the cell to form an expanded population. The exact conditions (e.g., temperature, CO₂ levels, agitation, presence of antibiotics, etc.) will depend on the other constituents of the medium and on the cell type. However, optimizing these parameters is within the ordinary skill in the art.

In another embodiment, the perineurium derived adult stem cells can be genetically modified, e.g., to express exogenous (e.g., introduced) genes (“transgenes”) or to repress the expression of endogenous genes, and the invention provides a method of genetically modifying such cells and populations. In accordance with this method, the perineurium derived adult stem cells is exposed to a gene transfer vector comprising a nucleic acid including a transgene, such that the nucleic acid is introduced into the cell under conditions appropriate for the transgene to be expressed within the cell. The transgene generally is an expression cassette, including a polynucleotide operably linked to a suitable promoter. The polynucleotide can encode a protein, or it can encode biologically active RNA (e.g., antisense RNA or a ribozyme). Thus, for example, the polynucleotide can encode a gene conferring resistance to a toxin, a hormone (such as peptide growth hormones, hormone releasing factors, sex hormones, adrenocorticotrophic hormones, cytokines (e.g., interferins, interleukins, lymphokines), etc.), a cell-surface-bound intracellular signaling moiety (e.g., cell adhesion molecules, hormone receptors, etc.), a factor promoting a given lineage of differentiation, (e.g., bone morphogenic protein (BMP)) etc. Of course, where it is desired to employ gene transfer technology to deliver a given transgene, its sequence will be known.

Within the expression cassette, the coding polynucleotide is operably linked to a suitable promoter. Examples of suitable promoters include prokaryotic promoters and viral promoters (e.g., retroviral ITRs, LTRs, immediate early viral promoters (IEp), such as herpesvirus IEp (e.g., ICP4-IEp and ICPO-IEp), cytomegalovirus (CMV) IEp, and other viral promoters, such as Rous Sarcoma Virus (RSV) promoters, and Murine Leukemia Virus (MLV) promoters). Other suitable promoters are eukaryotic promoters, such as enhancers (e.g., the rabbit β-globin regulatory elements), constitutively active promoters (e.g., the β-actin promoter, etc.), signal specific promoters (e.g., inducible promoters such as a promoter responsive to RU486, etc.), and tissue-specific promoters. It is well within the skill of the art to select a promoter suitable for driving gene expression in a predefined cellular context. The expression cassette can include more than one coding polynucleotide, and it can include other elements (e.g., polyadenylation sequences, sequences encoding a membrane-insertion signal or a secretion leader, ribosome entry sequences, transcriptional regulatory elements (e.g., enhancers, silencers, etc.), and the like), as desired.

The expression cassette containing the transgene should be incorporated into a genetic vector suitable for delivering the transgene to the cells. Depending on the desired end application, any such vector can be so employed to genetically modify the cells (e.g., plasmids, naked DNA, viruses such as adenovirus, adeno-associated virus, herpesviruses, lentiviruses, papillomaviruses, retroviruses, etc.). Any method of constructing the desired expression cassette within such vectors can be employed, many of which are well known in the art (e.g., direct cloning, homologous recombination, etc.). Of course, the choice of vector will largely determine the method used to introduce the vector into the cells (e.g., by protoplast fusion, calcium-phosphate precipitation, gene gun, electroporation, infection with viral vectors, etc.), which are generally known in the art.

The genetically altered perineurium derived adult stem cells can be employed as bioreactors for producing the product of the transgene. In other embodiments, the genetically modified perineurium derived adult stem cells are employed to deliver the transgene and its product to an animal. For example, the perineurium derived adult stem cells, once genetically modified, can be introduced into the animal under conditions sufficient for the transgene to be expressed in vivo.

In addition to serving as useful targets for genetic modification, populations of perineurium derived adult stem cells secrete hormones (e.g., cytokines, peptide or other (e.g., monobutyrin) growth factors, etc.). Some of the cells naturally secrete such hormones upon initial isolation, and other cells can be genetically modified to secrete hormones, as discussed herein. The perineurium derived adult stem cells that secrete hormones can be used in a variety of contexts in vivo and in vitro. For example, such cells can be employed as bioreactors to provide a ready source of a given hormone, and the invention pertains to a method of obtaining hormones from such cells. In accordance with the method, the perineurium derived adult stem cells are cultured, under suitable conditions for them to secrete the hormone into the culture medium. After a suitable period of time, and preferably periodically, the medium is harvested and processed to isolate the hormone from the medium. Any standard method (e.g., gel or affinity chromatography, dialysis, lyophilization, etc.) can be used to purify the hormone from the medium, many of which are known in the art.

In other embodiments, perineurium derived adult stem cells can be employed as therapeutic agents, for example in cell therapy applications. Generally, such methods involve transferring the cells to desired tissue, either in vitro (e.g., as a graft prior to implantation or engrafting) or in vivo, to animal tissue directly. The cells can be transferred to the desired tissue by any method appropriate, which generally will vary according to the tissue type. For example, perineurium derived adult stem cells can be transferred to a graft by bathing the graft (or infusing it) with culture medium containing the cells. Alternatively, the perineurium derived adult stem cells can be seeded onto the desired site within the tissue to establish a population. Cells can be transferred to sites in vivo using devices such as catheters, trocars, cannulae, stents (which can be seeded with the cells), etc. In one embodiment, the perineurium derived adult stem cell secretes a cytokine or growth hormone such as human growth factor, fibroblast growth factor, nerve growth factor, insulin-like growth factors, hemopoietic stem cell growth factors, members of the fibroblast growth factor family, members of the platelet-derived growth factor family, vascular and endothelial cell growth factors, members of the TGFb family (including bone morphogenic factor), or enzymes specific for congenital disorders (e.g., dystrophic).

In one application, the invention provides a method of promoting the closure of a wound within a patient using the cells of the invention. In accordance with the method, perineurium derived adult stem cells are transferred to the vicinity of a wound. The method promotes closure of both external (e.g., surface) and internal wounds. Wounds to which the present inventive method is useful in promoting closure include, but are not limited to, abrasions, avulsions, blowing wounds, burn wounds, contusions, gunshot wounds, incised wounds, open wounds, penetrating wounds, perforating wounds, puncture wounds, seton wounds, stab wounds, surgical wounds, subcutaneous wounds, or tangential wounds. The method need not achieve complete healing or closure of the wound; it is sufficient for the method to promote any degree of wound closure. In this respect, the method can be employed alone or as an adjunct to other methods for healing wounded tissue.

The perineurium derived adult stem cells of the invention can be employed in tissue engineering. In this regard, the invention provides a method of producing animal matter comprising maintaining the perineurium derived adult stem cells under conditions sufficient for them to expand and differentiate to form the desired matter. The matter can include mature tissues, or even whole organs, including tissue types into which the inventive cells can differentiate (as set forth herein). Typically, such matter will comprise adipose, cartilage, heart, dermal connective tissue, blood tissue, nervous tissue, muscle, kidney, bone, pleural, splanchnic tissues, vascular tissues, and the like. More typically, the matter will comprise combinations of these tissue types (i.e., more than one tissue type). For example, the matter can comprise all or a portion of an animal organ (e.g., a heart, a kidney) or a limb (e.g., a leg, a wing, an arm, a hand, a foot, etc.). Of course, in as much as the cells can divide and differentiate to produce such structures, they can also form anlagen of such structures. At early stages, such anlagen can be cryopreserved for future generation of the desired mature structure or organ.

To produce such structures, the perineurium derived adult stem cells are maintained under conditions suitable for them to expand and divide to form the desired structures. In some applications, this is accomplished by transferring them to an animal (i.e., in vivo) typically at a sight at which the new matter is desired. Thus, for example, the invention can facilitate the regeneration of tissues (e.g., bone, muscle, cartilage, tendons, adipose, etc.) within an animal where the perineurium derived adult stem cells are implanted into such tissues. In other embodiments the perineurium derived adult stem cells can be induced to differentiate and expand into tissues in vitro. In such applications, the perineurium derived adult stem cells are cultured on substrates that facilitate formation into three-dimensional structures conducive for tissue development. Thus, for example, the perineurium derived adult stem cells can be cultured or seeded onto a bio-compatible scaffold, such as one that includes extracellular matrix material, synthetic polymers, cytokines, growth factors, etc. In some embodiments, the perineurium derived adult stem cells are cultured along with one or more other cell populations, such as other stem cells, precursors, or mature cells. For example, the perineurium derived adult stem cells can be cultured on a biocompatible scaffold with osteoblasts or osteoprogenitor cells. In another example the perineurium derived adult stem cells are cultured on a biocompatible scaffold with neurons or neural progenitor cells. Such a scaffold can be molded into desired shapes for facilitating the development of tissue types.

Also, at least at an early stage during such culturing, the medium and/or substrate is supplemented with factors (e.g., growth factors, cytokines, extracellular matrix material, etc.) that facilitate the development of appropriate tissue types and structures. Indeed, in some embodiments, it is desired to co-culture the perineurium derived adult stem cells with mature cells of the respective tissue type, or precursors thereof, or to expose the cells to the respective conditioned medium, as discussed herein.

To facilitate the use of the perineurium derived adult stem cells for producing such animal matter and tissues, the invention provides a composition including the perineurium derived adult stem cells and biologically compatible scaffold. Typically, the scaffold is formed from polymeric material, having fibers as a mesh or sponge, typically with spaces on the order of between about 100 μm and about 300 μm. Such a structure provides sufficient area on which the cells can grow and proliferate. Desirably, the scaffold is biodegradable over time, so that it will be absorbed into the animal matter as it develops. Suitable polymeric scaffolds, thus, can be formed from monomers such as glycolic acid, lactic acid, propyl fumarate, caprolactone, hyaluronan, hyaluronic acid, and the like. Other scaffolds can include proteins, polysaccharides, polyhydroxy acids, polyorthoesters, polyanhydrides, polyphosphazenes, or synthetic polymers (particularly biodegradable polymers). Of course, a suitable polymer for forming such scaffolds can include more than one monomers (e.g., combinations of the indicated monomers). Also, the scaffolds can also include hormones, such as growth factors, cytokines, and morphogens (e.g., retinoic acid, aracadonic acid, etc.), desired extracellular matrix molecules (e.g., fibronectin, laminin, collagen, etc.), or other materials (e.g., DNA, viruses, other cell types, etc.) as desired.

To form the composition, the perineurium derived adult stem cells are introduced into the scaffold such that they permeate into the interstitial spaces therein. For example, the matrix can be soaked in a solution or suspension containing the cells, or they can be infused or injected into the matrix. An exemplary composition is a hydrogel formed by crosslinking of a suspension including the polymer and also having the inventive cells dispersed therein. This method of formation permits the cells to be dispersed throughout the scaffold, facilitating more even permeation of the scaffold with the cells. Of course, the composition also can include mature cells of a desired phenotype or precursors thereof, particularly to potentate the induction of the perineurium derived adult stem cells to differentiate appropriately within the scaffold (e.g., as an effect of co-culturing such cells within the scaffold). In one embodiment, the perineurium derived adult stem cells acts as a support cell population to enhance the cell growth, proliferation, differentiation, etc. of mature cells or precursors thereof.

The composition can be employed in any suitable manner to facilitate the growth and generation of the desired tissue types, structures, or anlagen. For example, the composition can be constructed using three-dimensional or stereotactic modeling techniques. Thus, for example, a layer or domain within the composition can be populated by cells primed for osteogenic differentiation, and another layer or domain within the composition can be populated with cells primed for myogenic and/or chondrogenic development. Bringing such domains into juxtaposition with each other facilitates the molding and differentiation of complex structures including multiple tissue types (e.g., bone surrounded by muscle, such as found in a limb). To direct the growth and differentiation of the desired structure, the composition can be cultured ex vivo in a bioreactor or incubator, as appropriate. In other embodiments, the structure is implanted within the host animal directly at the site in which it is desired to grow the tissue or structure. In still another embodiment, the composition can be engrafted on a host (typically an animal such as a pig, baboon, etc.), where it will grow and mature until ready for use. Thereafter, the mature structure (or anlagen) is excised from the host and implanted into the host, as appropriate.

Scaffolds suitable for inclusion into the composition can be derived from any suitable source (e.g., matrigel), and some commercial sources for suitable scaffolds exist (e.g., suitable of polyglycolic acid can be obtained from sources such as Ethicon, N.J.). Another suitable scaffold can be derived from the acellular tissue—i.e., tissue extracellular matrix matter substantially devoid of cells, and the invention provides such a acellular derived scaffold. Typically, such acellular derived scaffolds includes proteins such as proteoglycans, glycoproteins, hyaluronins, fibronectins, collagens (type I, type II, type imi, type IV, type V, type VI, etc.), and the like, which serve as excellent substrates for cell growth. Additionally, such acellular derived scaffolds can include hormones, preferably cytokines and growth factors, for facilitating the growth of cells seeded into the matrix.

Tissue-derived matrix can be isolated from tissue. For example, tissue can be subjected to sonic or thermal energy and/or enzymatic processed to recover the matrix material. Also, desirably the cellular fraction of the tissue is disrupted, for example by treating it with lipases, detergents, proteases, and/or by mechanical or sonic disruption (e.g., using a homogenizer or sonicator). However isolated, the material is initially identified as a viscous material, but it can be subsequently treated, as desired, depending on the desired end use. For example, the raw matrix material can be treated (e.g., dialyzed or treated with proteases or acids, etc.) to produce a desirable scaffold material. Thus the scaffold can be prepared in a hydrated form or it can be dried or lyophilized into a substantially anhydrous form or a powder. Thereafter, the powder can be rehydrated for use as a cell culture substrate, for example by suspending it in a suitable cell culture medium. In this regard, the acellular derived scaffold can be mixed with other suitable scaffold materials, such as described above. Of course, the invention pertains to compositions including the acellular derived scaffold and cells or populations of cells, such as the inventive perineurium derived adult stem cells and other cells as well (particularly other types of stem cells).

As discussed above, the perineurium derived adult stem cells, populations, scaffolds, and compositions of the invention can be used in tissue engineering and regeneration. Thus, the invention pertains to an implantable structure (i.e., an implant) incorporating any of these inventive features. The exact nature of the implant will vary according to the use to which it is to be put. The implant can be or comprise, as described, mature tissue, or it can include immature tissue or the scaffold. Thus, for example, one type of implant can be a bone implant, comprising a population of the inventive cells that are undergoing (or are primed for) osteogenic differentiation or are supporting osteogenic differentiation, optionally seeded within a scaffold of a suitable size and dimension, as described above. Such an implant can be injected or engrafted within a host to encourage the generation or regeneration of mature bone tissue within the patient. Similar implants can be used to encourage the growth or regeneration of muscle, fat, cartilage, tendons, etc., within patients. Other types of implants are anlagen (such as described herein), e.g., limb buds, digit buds, developing kidneys, etc, that, once engrafted onto a patient, will mature into the appropriate structures.

In one embodiment, the perineurium derived adult stem cells of the invention are used to induce and support regeneration and repair of neuronal tissue. The cells of the invention can promote neuroregeneration, including but not limited to axonal regeneration, neuronal regeneration, and peripheral nerve regeneration. For example, the cells can be used in methods to treat spinal cord injury, peripheral neuropathy, neurodegenerative disorders, neuropathic pain, and the like. It is described elsewhere herein, that the perineurium derived adult stem cells and cells derived thereof express neural guidance molecules reelin and VEGF-D. As such, in one embodiment, the cells of the invention can be administered to a site in need of neuroregeneration. In one embodiment, a biocompatible scaffold comprising the cells of the invention is implanted at a site in need of neuroregeneration. Non-limiting examples of such a scaffold includes a nerve guidance conduit, hydrogel, electrospun scaffold, foam, mesh, and sponge. The perineurium derived adult stem cells comprised scaffold can further contain neurons, astrocytes, microglia, oligodendrocytes, Schwann cells, neural progenitor cells, and a combination thereof. Further the perineurium derived adult stem cells comprised scaffold can contain growth factors, neuronal guidance cues, neurotransmitters, extracellular matrix components, and the like. Non-limiting examples of neuronal guidance cues include netrins, epherins, cell adhesion molecules, BMPs, Wnts, and growth factors. Such molecules or compounds can be dispersed throughout the scaffold. In one embodiment, such molecules or compounds are adhered to microspheres or nanospheres dispersed throughout the scaffold. In one embodiment, guidance cues are located at distinct locations within the scaffold, thereby guiding the directed growth of the neuron. In one embodiment, cells, including the cells of the invention, are modified to express proteins that act as guidance cues. Such proteins can be expressed on the surface of the modified cell or alternatively can be secreted by the modified cell.

In one embodiment the cells of the invention control the microenvironmental oxygen tension, thereby allowing an environment beneficial for chondrogenesis, lymphangiogenesis, neurite outgrowth, osteogenesis, and the like. For example, the cells of the invention promote hypoxia and neovascularization. In one embodiment, the cells of the invention are used to co-operate with Schwann cells in methods to form either a lipid coat, myelin, or both on newly made nerves in vivo or ex vivo. In another embodiment, the cells of the invention are used to co-operate with Schwann cells in methods to remyelinate existing nerves.

In one embodiment, the cells of the invention are used methods to treat disorders such as obesity, diabetes, and metabolic syndrome. Cells of the invention can regulate triglyceride homeostasis, and thus can be used in cell therapy applications to help combat such disorders. The cells can be administered to a site within a subject by any method known in the art, as described elsewhere herein.

In one embodiment, the cells of the invention are used in methods to treat cancer. Cells of the invention are ADRB3+. Prior research has shown that mutations in ADRB3 can bring susceptibility to cancer (Huang et al., 2001, BCR, 3: 264-269). In one embodiment, the cells of the invention can be used in cell therapy or tissue engineering applications to treat various types of cancers, including but not limited to breast cancer, lung cancer, pancreatic cancer, osteosarcoma, neuroblastoma, lymphomas, leukemias, prostate cancer, bone cancer, neurofibromatosis, and brain cancer.

In some embodiments, the cells of the invention are stimulated with BMP2, noradrenaline, or a combination thereof. In one embodiment, cells are administered to a subject and the subject is either systemically or locally stimulated with BMP2, noradrenaline, or a combination thereof. For example, the subject can be injected at a particular site with BMP2, noradrenaline, or a combination thereof. In another embodiment, the cells of the invention are stimulated with BMP2, noradrenaline, or a combination thereof in an in vitro or ex vivo environment prior to administering the cells to the subject.

While many aspects of the invention pertain to tissue growth and differentiation, the invention has other applications as well. For example, the perineurium derived adult stem cells seeded scaffold can be used as an experimental reagent, such as in developing improved scaffolds and substrates for tissue growth and differentiation. The scaffold also can be employed cosmetically, for example, to hide wrinkles, scars, cutaneous depressions, etc., or for tissue augmentation. For such applications, preferably the scaffold is stylized and packaged in unit dosage form. If desired, it can be admixed with carriers (e.g., solvents such as glycerin or alcohols), pharmaceuticals, vitamins, therapeutic proteins, and the like. The substrate also can be employed autologously or as an allograft, and it can be used as, or included within, ointments or dressings for facilitating wound healing. The perineurium derived adult stem cells can also be used as experimental reagents. For example, they can be employed to help discover agents responsible for early events in differentiation. For example, the inventive cells can be exposed to a medium for inducing a particular line of differentiation and then assayed for differential expression of genes (e.g., by random-primed PCR or electrophoresis or protein or RNA, etc.).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Brown Adipocyte-Like Cells (BALCs) Derived from Peripheral Nerves in Response to BMP2

One of the earliest in vivo responses to BMP2 delivery in skeletal muscle is the rapid, but transient, appearance of brown adipocyte-like cells (BALCs). BALCs can rapidly lower local oxygen tension during aerobic respiration through uncoupling the ATP synthase step of electron transport by uncoupling protein 1 (UCP1), resulting in heating that further reduces oxygen tension. Recent studies suggest that BALCs are generated in response to noradrenaline binding to β-adrenergic receptors 3 (ADRB3) on precursors after activation of the sympathetic nervous system (SNS). In these studies, AdBMP2 transduced cells were injected into skeletal muscle, which led to a significant elevation in noradrenaline within the mouse circulation 48 hours later. Changes in noradrenaline were followed by an increase in ADRB3+ cells within the perineurial region of peripheral nerves surrounding the site of BMP2 delivery Immunohistochemical staining showed that a subset of these cells were replicating within 2 days after exposure to BMP2. FACS analysis of cells isolated from the sciatic nerve, adjacent to the BMP2 delivery site, showed significantly more ADRB3+ cells. Surprisingly, similar analysis performed on cells isolated from the nerve 4 days post BMP2 delivery, revealed a significant decrease in ADRB3+ cells. Simultaneously with this decrease, FACS analysis of the soft tissues surrounding the site of BMP2 delivery revealed a significant increase suggesting that the cells had migrated from the perineurial region of the nerve towards the BMP2. Further analysis confirmed that the ADRB3+ cell population within soft tissues also expressed UCP1. Quantification of ADRB2, ADRB3, and UCP1-specific RNAs in the tissues, revealed a significant increase in both ADRB3 and UCP1 RNA levels, by 3-4 days after injection of the AdBMP2 transduced cells, whereas ADRB2 RNA levels remained unchanged. To further confirm that these cells were migrating from the perineurium, nerve remodeling was blocked through delivery of cromolyn, which completely ablated of brown adipogenesis. The data presented herein collectively suggests that BALC precursors reside in the peripheral nerves and expand/migrate/differentiate in response to BMP2. BALCs also expressed the HNK1 carbohydrate epitope, a marker for neural stem cell migration. Suprisingly, BALCs also synthesized the neural guidance molecule reelin. Therefore, BALCs with their combined activities in regulation of oxygen tension, neovascularization, and neural guidance, are likely a key director of tissue formation in the adult.

The materials and methods employed in these experiments are now described.

Heterotopic Bone Assay

Murine fibroblast cells were transduced with either AdBMP2 or Adempty cassette control virus at a concentration of 5000 vp/cell with 1.2% GeneJammer (Dilling et al., 2010, J Bone Miner Res 25:1147-1156; Fouletier-Dilling et al., 2005, Hum Gene Ther 16:1287-1297; Gugala et al., 2003, Gene Ther 10:1289-1296; Olmsted-Davis et al., 2002, Human Gene Therapy 13:1337-1347). Replication defective E1-E3 deleted first generation human type 5 adenovirus possessing cDNA for BMP2 (AdBMP2) or no transgene (Adempty) were constructed as previously described (Olmsted-Davis et al., 2002, Human Gene Therapy 13:1337-1347).

The transduced cells were resuspended at a concentration of 5×10⁶ cells/1000 μL of PBS and delivered through an intramuscular injection into the hind limb quadriceps muscle of C57/BL6 mice (Jackson Laboratories, Bar Harbor Me.). Animals were euthanized at daily intervals after injection as indicated in the text. Hind limbs or sciatic nerves were harvested and either placed in formalin or quick frozen and stored at −80° C. All animal studies were performed in accordance with standards of Baylor College of Medicine, Department of Comparative Medicine, after review and approval of the protocol by the Institutional Animal Care and use Committee (IACUC).

Cromolyn Administrations

Intraperitoneal injections of sodium cromoglycate (C0399, Sigma-Aldrich, St. Louis Mo.) were administered daily (8 mg/kg/day) for five days prior to intramuscular injection of transduced cells, and then continued daily throughout the time course of the heterotopic bone assay, as previously described (Salisbury et al., 2011, J Cell Biochem 112(10):2748-58). Animals were euthanized at specified time points following injection of transduced cells.

Quantification of Noradrenaline Levels

Blood was collected from animals (n=3) receiving intramuscular injection of either AdBMP2 or Adempty transduced cells. Plasma was separated by centrifugation at 1000×g for 15 minutes at 4° C. Quantification of noradrenaline levels were assayed by ELISA (Cat No. 40-734-35002, GenWay, San Diego Calif.) according to the manufacturer's protocol. Sample analysis was done in duplicate, the results from each day following injection were averaged, and significance was determined by Student's t-test.

Q-RT-PCR (Quantitative Real-Time Reverse Transcriptase Polymerase Chain Reaction)

Total RNA from the entire hind limb soft tissues that received AdBMP2 or Adempty transduced cells were extracted using a TRIZOL Reagent (Life Technologies, Carlsbad, Calif.) and purified using the Qiagen RNeasy Mini Kit, according to the manufacturer's protocol for RNA clean-up (Qiagen, Valencia Calif.). Muscle samples (n=4) were collected every day, for 6 days following injection. RNA integrity was confirmed by agarose gel electrophoresis and concentrations were determined spectrophotometrically. The cDNA was synthesized from RNA using the RT2 first strand kit (SA Biosciences Inc, Fredrick Md.). Real time qPCR analyses were done using RT2 qPCR Primer Assay (SA Biosciences Inc, Fredrick, Md.) for ADRB3 (Cat. PPM04810E-200), ADRB2 (Cat. PPM04265B-200), and UCP1 (Cat. PPM05164A-200). For normalization, Tbp (TATA box binding protein, Cat. PPM03560E-200) was found to be the best internal control. The RT2 SYBR Green/ROX Master Mix (SA Biosciences Inc) was used for PCR amplification. The cDNA was subjected to qRT-PCR analyses in parallel using a 7900HT PRISM Real-Time PCR machine and SDS 2.3 software (Applied Biosystems, Carlsbad Calif.). The Ct values were determined for each biological sample in duplicate, normalized against Tbp as an internal control, and expressed in relation to RNA isolated from control tissues as a calibrator. Relative gene expression was determined using the AA Ct method and the results for either control or BMP2 tissues at each time point were averaged and the standard error of the mean calculated. Statistical significance was determined by the Student's t-test. For comparison studies of RNA expression in cromolyn treated animals exposed to BMP2, relative gene expression was again determined using the same ΔΔ Ct method, but in this instance Ct values were expressed in relation to vehicle treated animals injected after exposure to AdBMP2 transduced cells.

Isolation of Sciatic Nerve and Muscle Cells

Sciatic nerves were dissected and cells isolated following previously described methods (Bixby et al., 2002, Neuron 35:643-656). Briefly, sciatic nerves were dissected into cold Ca²⁺, Mg²⁺ free Hank's buffered salt solution (HBSS) and dissociated by incubating for 4 minutes at 37° C. in trypsin-versene (EDTA) diluted 1:10 in Ca²⁺, Mg²⁺ free HBSS, plus 0.25 mg/mL type 4 collagenase (Worthington, Lakewood N.J.). After centrifugation, nerve cells were triturated, filtered through nylon mesh, and resuspended in cell staining buffer (Biolegend, SanDiego, Calif.). Quadriceps muscle tissue was dissected from the skeletal bone into cold HBSS and dissociated by mincing the tissues and incubating for 45 minutes at 37° C. in 0.2% type 2 collagenase (Worthington, Lakewood N.J.) in HBSS. An equal volume of Dulbecco's modified eagle medium supplemented with 10% FBS was added to quench the digestion reaction, the cells were centrifuged, triturated, filtered through nylon mesh and resuspended in cell staining buffer. In each experiment, the sciatic nerves or muscles isolated from 3 animals (for a total of 6 nerves or muscles) were pooled for further staining and FACS analysis.

Flow Cytometry and FACS (Fluorescence Activated Cell Sorting)

Cells isolated from either the sciatic nerve or muscle were incubated with ADRB3 antibody (chicken polyclonal, ab59685, Abcam Incorporated, Cambridge Mass., 1:400 dilution) for 45 minutes on ice. Cells were washed with PBS and then incubated with anti-chicken Alexa Fluor 488 (Invitrogen, Carlsbad Calif., 1:500 dilution) for 30 minutes on ice. Cells were again washed with PBS and stained cells were analyzed on a FACSAriaII (BD, Becton Dickson, Mountain View Calif.) flow cytometer and BD FACSDiva Software. For cell sorting, labeled cells were separated based on their fluorescence intensity and the ADRB3 negative and positive population were collected with >95% purity. Cytospin slide preparations of the sorted cells were produced by centrifugation of approximately 40,000 cells at 500 rpm for 5 minutes. The slides were subsequently stained with an antibody to UCP1, according to the immunostaining methods below. The percentage of positive cells from each experiment was averaged, the standard error of the mean was calculated, and statistical significance determined by the Student's t-test.

Immunohistochemical and Immunocytochemical Analysis

Mouse hind limbs were isolated, formalin fixed, decalcified, and processed for paraffin sectioning. Prior to sectioning the tissues were cut and both halves embedded so that the tissues are sectioned from the inside outward as previously described (Olmsted-Davis et al., 2007, Am J Pathol 170:620-632). Serial sections were prepared (5 μm) and every fifth slide stained with hematoxylin and eosin. Serial unstained slides were used for immunohistochemical staining (either single- or double-antibody labeling), using methods outlined previously (Olmsted-Davis et al., 2007, Am J Pathol 170:620-632). Briefly, for immunofluorescence staining, samples were incubated with primary antibodies, followed by washing and incubation with respective secondary antibodies, used at 1:500 dilution, to which Alexa Fluor 488, 594, or 647 (Invitrogen, Carlsbad Calif.) was conjugated. Primary antibodies were used as follows: UCP1, rabbit polyclonal, used at 1:200 dilution (Chemicon, Temecula Calif.), ADRB3, rabbit polyclonal, used at 1:100 dilution (Cell Applications Inc, San Diego Calif.), neurofilament, mouse monoclocal, used at 1:200 dilution (Sigma, St Louis Mo.), and Ki67, rat monoclonal, used at 1:50 dilution (Dako, Carpinteria Calif.). Primary and secondary antibodies were either diluted in 2% bovine serum albumin (BSA), or for mouse primary antibodies staining was performed using the Mouse on Mouse (M.O.M.) kit (Vector Labs, Burlingame Calif.) according to the manufacturer's protocol. Tissues were counterstained and covered with Vectashield mounting medium containing DAPI (Vector Laboratories, Burlingame Calif.). When sections were stained using horseradish peroxidase, they were analyzed with the PowerVision Poly-HRP anti-Rabbit IHC Detection System (Leica Microsystems, Buffalo Grove Ill.), according to manufacturer's instructions and were counterstained with hematoxylin.

Mouse sciatic nerves were frozen sectioned on a longitudinal plane and fixed with 4% paraformaldehyde, PBS washed and treated with 0.3% Trition X-100 in Tris-buffered saline, and subsequently stained following the immunofluorescence procedures described above.

Cytospin preparations were immunostained following similar methods. Briefly, cells were fixed with 4% paraformaldehyde, PBS washed, treated with 0.3% Trition X-100 in 0.3% Tris-buffered saline, blocked with 2% BSA, and incubated in primary antibody overnight. After PBS washing, samples were incubated in the appropriate secondary antibody and counterstained with DAPI.

The results of the experiments are now described.

Up-Regulation of Sympathetic Activity Prior to Induction of BALCs after Exposure to BMP2

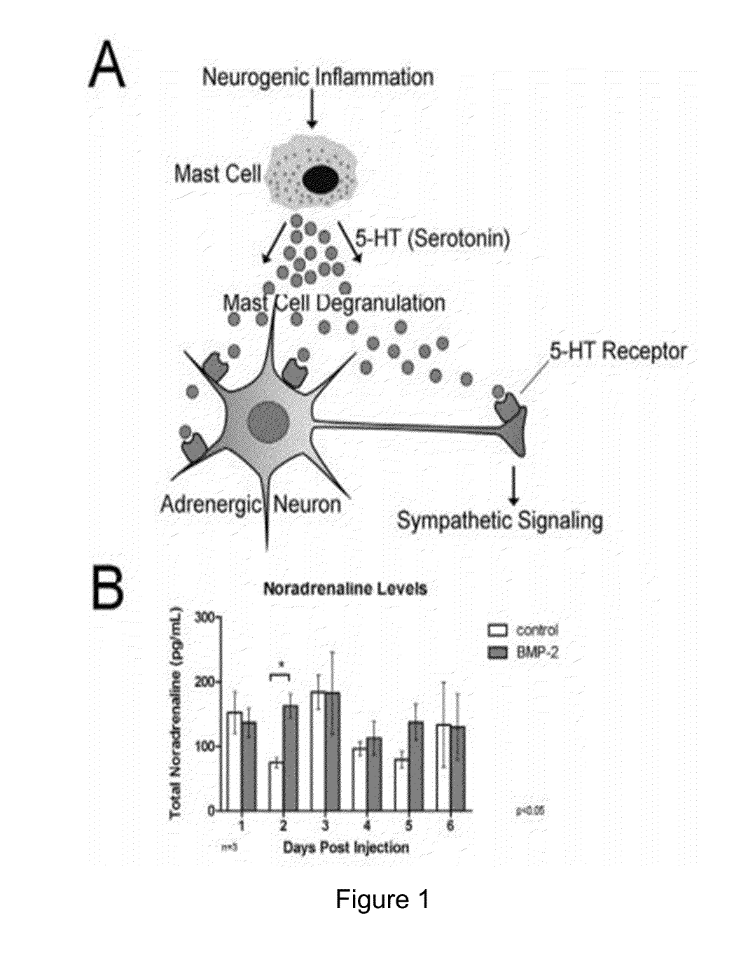

A critical role for neurogenic inflammation and subsequent mast cell degranulation in the formation of heterotopic bone was previously demonstrated (Salisbury et al., 2011, J Cell Biochem 112:2748-2758). Mast cells can release serotonin upon degranulation, leading to sympathetic nerve activation (FIG. 1A) (Nichols and Nichols, 2008, Chem Rev 108:1614-1641; Ries and Fuder, 1994, Methods Find Exp Clin Pharmacol 16(6):419-435; Theoharides et al., 1982, Nature 297:229-231). One of the first steps in SNS signaling is the release of noradrenaline which is synthesized and released by noradrenergic nerves. Therefore the levels of this hormone were measured after exposure to BMP2. A significant increase (p<0.05) in noradrenaline was observed in animals receiving the AdBMP2 transduced cells, as compared to controls, animals receiving Adempty transduced cells, 2 days following induction of heterotopic bone formation (FIG. 1B). The data suggests enhanced sympathetic activity in the initial days following BMP2 stimulation.

Noradrenaline Induces Replication of ADRB3+Cells Localized within the Perineurial Region of Peripheral Nerves after Exposure to BMP2

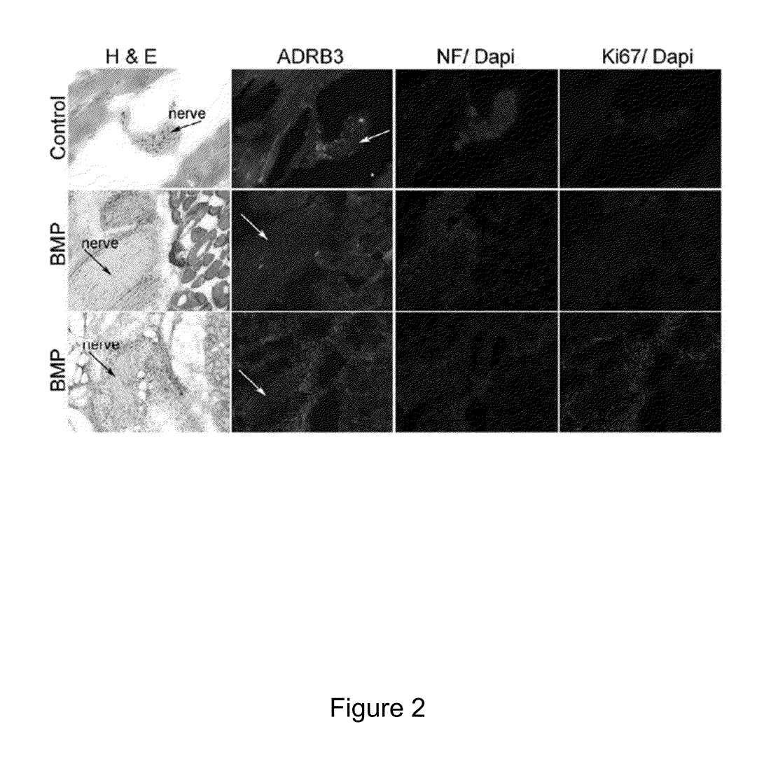

Noradrenaline released from sympathetic nerves has previously been found to bind and activate adrenergic receptor initiating a signaling cascade that ultimately leads to the expression of UCP1 (Cannon and Nedergaard, 2004, Physiol Rev 84:277-359; Lowell and Spiegelman, 2000, Nature 404:652-660; Klingenspor, 2003, Exp Physiol 88:141-148). To determine what cells within the skeletal muscle possess adrenergic receptor, mouse hind limb tissues receiving either AdBMP2 or Adempty transduced cells, were isolated at 1 and 3 days after injection and immunostained for ADRB3+ cells (FIG. 2). Analysis of serial sections isolated from the tissues receiving the Adempty cells, showed positive expression (red color) of this receptor on cells within the perineurial region of peripheral nerves, as determined by expression of neurofilament (green color), but a lack of positive expression in skeletal muscle or white adipose tissue (FIG. 2). Alternatively, tissues isolated from mice exposed to BMP2 for 1 and 2 days also had similar positive expression in the nerve, but ADRB3+ cells were also observed interspersed between skeletal muscle and adipocytes in the day 3 tissues (FIG. 2) and these cells were replicating as evidenced by labeling with Ki67.

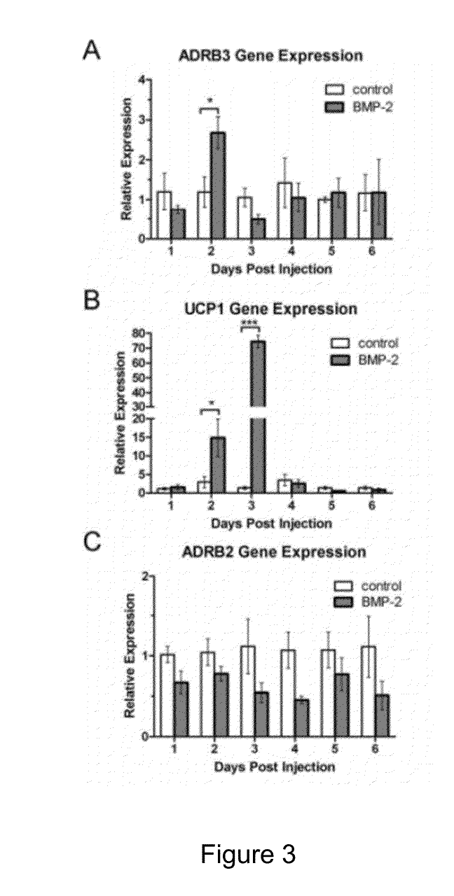

Quantitative reverse transcription PCR was performed for ADRB2, ADRB3, and UCP1-specific RNA was performed on a daily basis for 6 days on soft tissues encompassing the region of BMP2 expression. Soft tissues isolated at day 2 after injection of BMP2-producing cells, had a significant increase (p<0.05) in ADRB3, but not ADRB2-specific RNA as compared to the tissues receiving control transduced cells isolated in parallel (FIG. 3). Additionally, UCP1-specific RNA was dramatically elevated on both days 2 and 3 with a greater than 70-fold increase on day 3 (p<0.0005) that fell to background levels on day 4 (FIG. 3).

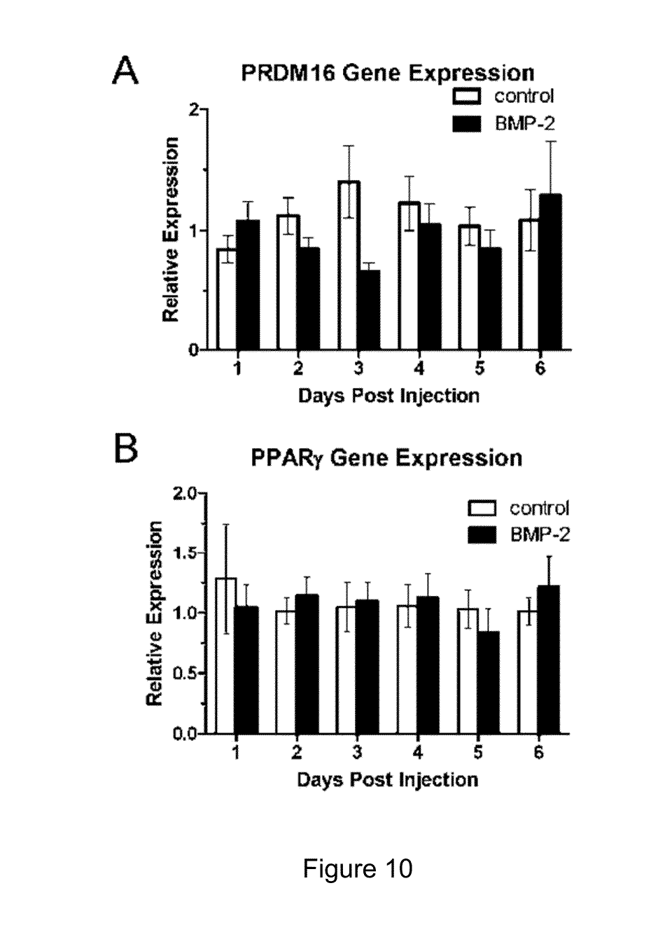

While ADRB3 is the most predominant effector of adrenergic stimulation on BAT biogenesis, β2-adrenergic receptor may also participate (Cannon and Nedergaard, 2004, Physiol Rev 84:277-359; Bachman et al., 2002, Science 297:843-845). In addition, β2-adrenergic receptor (ADRB2) signaling has been implicated in the regulation of bone mass (Takeda and Karsenty, 2008, Bone 42:837-840). Therefore, RNA expression of ADRB2 was also examined, which was found not to be significantly changed in the tissues of animals receiving BMP2-producing cells, compared to that of the control tissues (FIG. 3C). Several genes have been associated with the generation of brown adipose tissue, so thus these RNAs were analyzed for changes in these other genes. Surprisingly, significant changes were not observed in expression of other genes reported to be associated with BAT biogenesis, including PRDM16, PPARγ, PPARα, PPARΔ, (FIG. 10), although PGC1α had a trend toward increase but this change was not found to be significant. The data suggests that this transient brown adipose tissue may be different from other brown adipose tissue previously described.

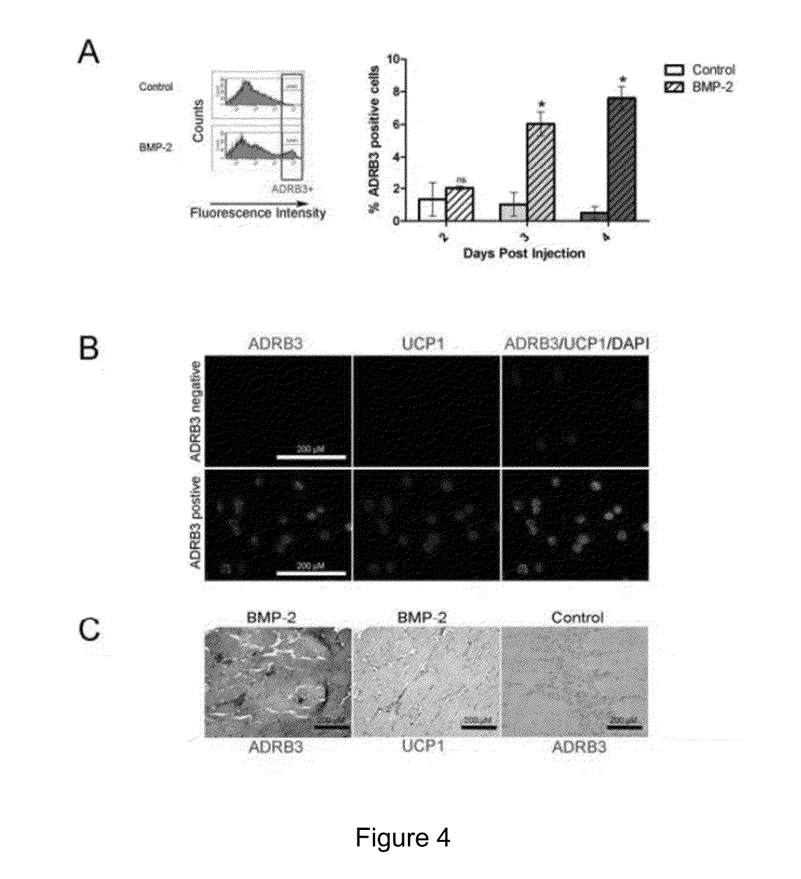

The increase in ADRB3 in the injected area was quantified by FACS analysis for ADRB3+ cells on days 2, 3, and 4 (FIG. 4). The percentage of positive cells was increased on days 3 and 4, but a similar increase was not observed after injection of a similar number of cells transduced with Adempty (FIG. 4). When isolated ADRB3+ cells were subjected to analysis for UCP1, almost all cells were positive (FIG. 4), indicating that many of these cells had acquired the phenotype of brown adipocytes.

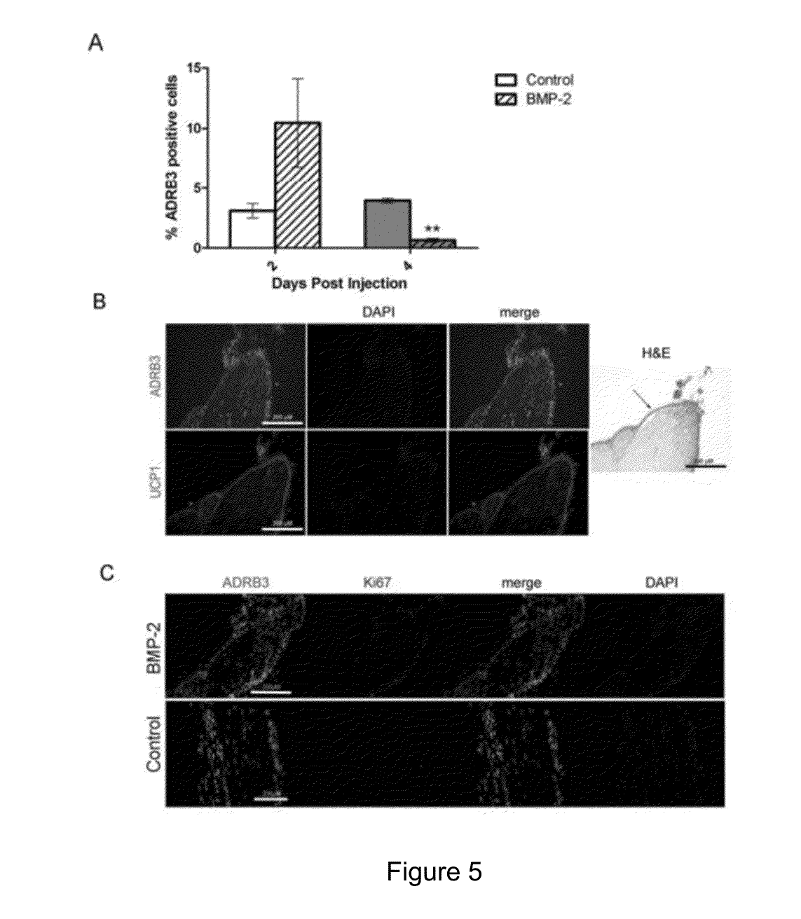

Similar flow cytometric analysis was also performed on cells isolated from the sciatic nerves. In contrast to the entire soft tissues, a marked increase in the percentage of ADRB3+ cells (FIG. 5A) was seen in populations isolated from the sciatic nerve after exposure to BMP2 for 2 days. Surprisingly, expression of ADRB3 was dramatically decreased (p<0.05) within cells of the sciatic nerve 4 days following injection of AdBMP2 transduced cells, as compared to controls. Again this was in contrast to the increase in ADRB3+ cells interspersed within the skeletal muscle (FIG. 4). The data collectively suggests that ADRB3+ cells within the peripheral nerve leave the nerve, expand, and migrate towards the BMP2. To test this it was next determined if the ADRB3+ cell populations were undergoing replication and migration.

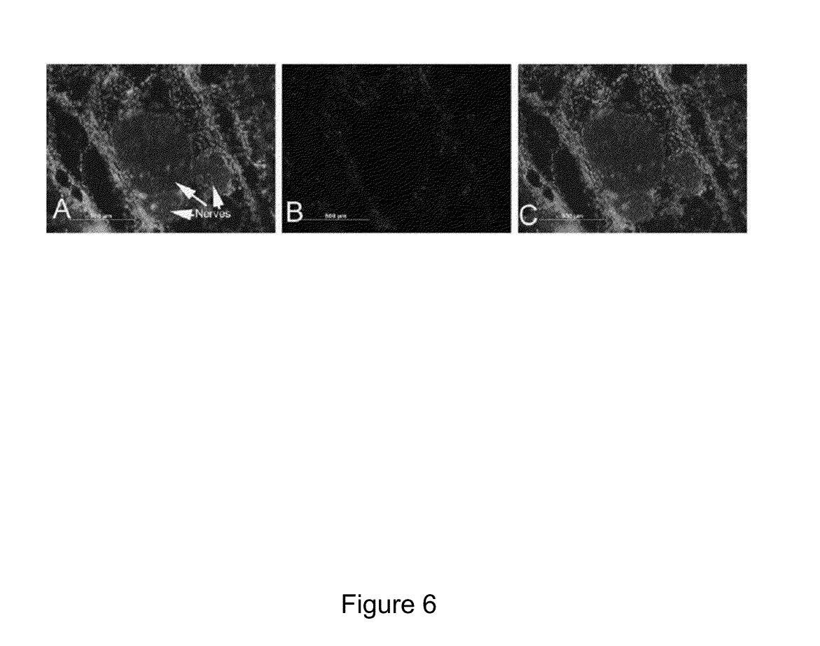

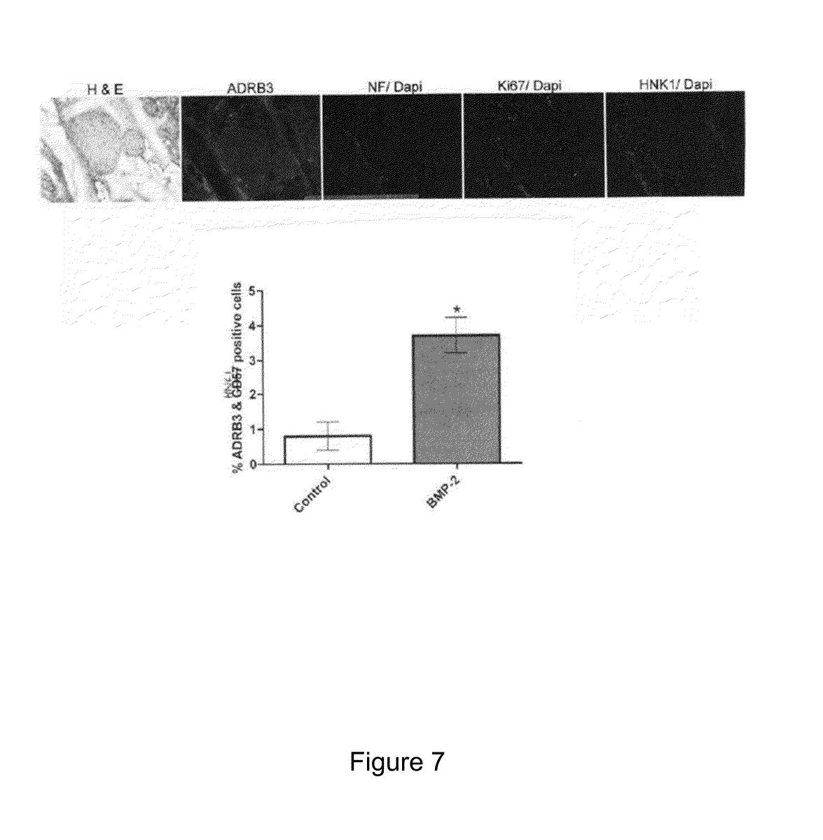

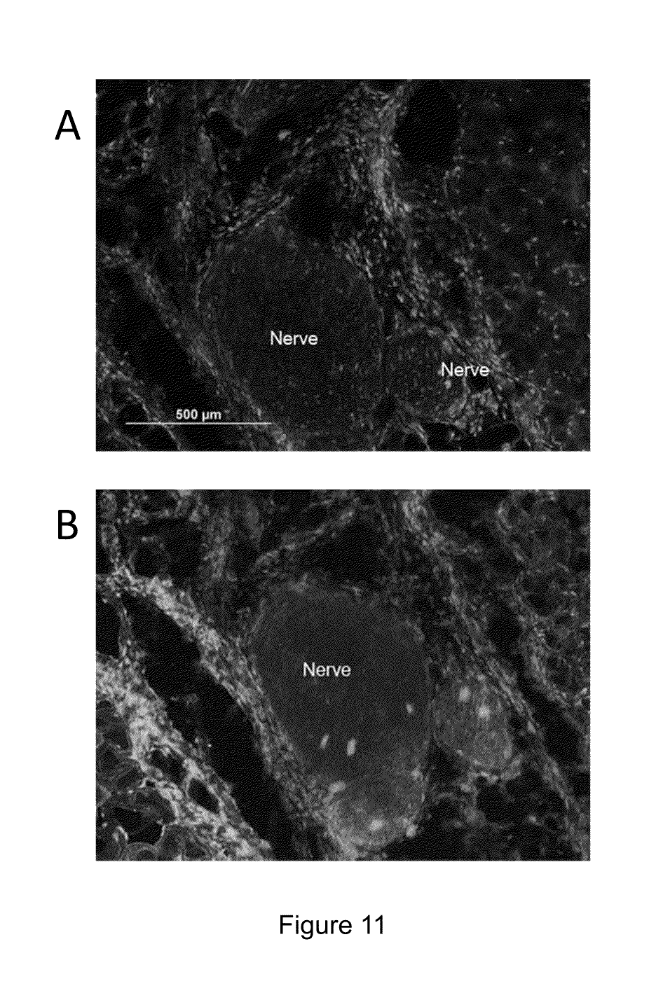

To determine whether the cells were replicating, the same nerve tissue sample was immunostained for expression of the cellular replication marker Ki67. FIG. 5C shows co-localization of ADRB3 (green) and Ki67 (red) in sciatic nerve tissue of BMP2 treated animals. These proliferating cells again appeared localized to the perineurium as well as cells outside, but in close proximity, to the nerve. FIG. 6 shows expression of ADRB3 (green) and Ki67 (red) in other nerves on the third day after BMP2 induction. In FIG. 6 it is notable that almost all cells expressing the replication marker Ki67 (FIG. 6B), also express ADBR3 (FIG. 6A). Since it is likely that there are progenitors for cells other than brown adipocytes after BMP2 induction, including osteoblasts (Salisbury et al., 2011, J Cell Biochem 112(10):2748-58), it may mean that this receptor plays a more general role in differentiation of progenitors derived from peripheral nerves. There was little to no replication observed within the nerve tissue of control animals, suggesting that the ADRB3+ cells within the nerve were responding to the changes induced by BMP2, to undergo expansion. To determine if these cells were also capable of migrating from the nerve, immunostaining for the neuronal migratory marker (Kunemund et al., 1988, J Cell Biol 106:213-223; Nagase et al., 2001, Dev Growth Differ 43:683-692; Jungalwala et al., 1994, Neurochem Res 19:945-957) HNK1 was performed in the peripheral nerve tissues. The results showed that subsets of the ADBR3+(green) cells expressed the HNK 1 carbohydrate epitope (red, FIG. 7). This marker was expressed strongly in cells inside nerves from BMP2 induced tissue (FIG. 7) and less strongly in tissue after injection with cells transduced with empty vector. A similar staining pattern was observed for beta-1,3-gluconoryl transferase 2 (B3GAT2), one of the enzymes involved in the formation of HNK (Yamamoto et al., 2002, J Biol Chem 277:27227-27231) (FIG. 11). In addition to co-staining within the perineurial region, HNK also stained around the endoneurium around the axon both with and without BMP2 induction (FIG. 11). The expression of HNK in this region was not accompanied by ADRB3 co-staining

To quantify the potential increase expression of HNK1 after exposure to BMP2, and confirm co-expression on ADRB3+ cells, soft tissues from mouse hind limb 4 days after initial delivery of BMP2 was subjected to FACS analysis. This analysis also shows that the ADRB3+ population also expresses the HNK1 migration marker (FIG. 11). The data collectively suggests that cells expressing ADBR3 in the perineurial region of peripheral nerves undergo replication and migration from the nerve towards the location of BMP2 expression.

ADBR3+Cells Differentiate into BALCs

Immunohistochemical analysis of the sciatic nerve tissue sections isolated 2 days after delivery of AdBMP2 transduced cells revealed expression of ADRB3 (green) on the nerve, seemingly co-aligning with UCP1 expression (green) of a serial tissue section (FIG. 5B). These tissue sections were cut on a longitudinal plane, exposing the perineurial layer of the nerve in the location indicated in FIG. 5B. Intriguingly, many of these ADRB3 and UCP1 positive cells appeared within this perineurial region, although there were also positive cells located within inner layers of the nerve tissue. Consistent with the flow cytometry results, ADRB3 and UCP1 expression analyzed by immunohistochemistry 4 days after receiving AdBMP2 transduced cells was also minimally observed co-aligning within the nerve tissues (data not shown). Instead, as shown in FIG. 3C, at this time point, ADRB3 and UCP1 expression appeared to overlap in cells within the muscle. While ADRB3 expression within the muscle was almost exclusively aligned with UCP1 on day 4, there were some ADRB3 positive cells which did not co-align with UCP1 expression on day 2 within the nerve.

To confirm the co-expression of UCP1 within these cells, ADRB3+ cells were isolated after 4 days of exposure to BMP2, centrifuged onto slides, and immunostained for the brown adipocyte specific marker UCP1. As illustrated in FIG. 4B, the ADRB3 positive cells (green) co-expressed UCP1 (red), while the ADRB3 negative population showed minimal to no staining for UCP1. This data suggests an induction of brown adipocytes, which express both ADRB3 and UCP1, within the muscle soft tissues surrounding the site where new bone is forming 4 days following delivery of the AdBMP2 transduced cells. Collectively, this data suggests that ADRB3 positive progenitors may reside within the perineurial region of peripheral nerves, where upon SNS signaling causes them to expand and differentiate into brown adipocytes.

### Inhibition of Sympathetic Pathway by Blocking Mast Cell Degranulation Suppresses Induction of BALCs

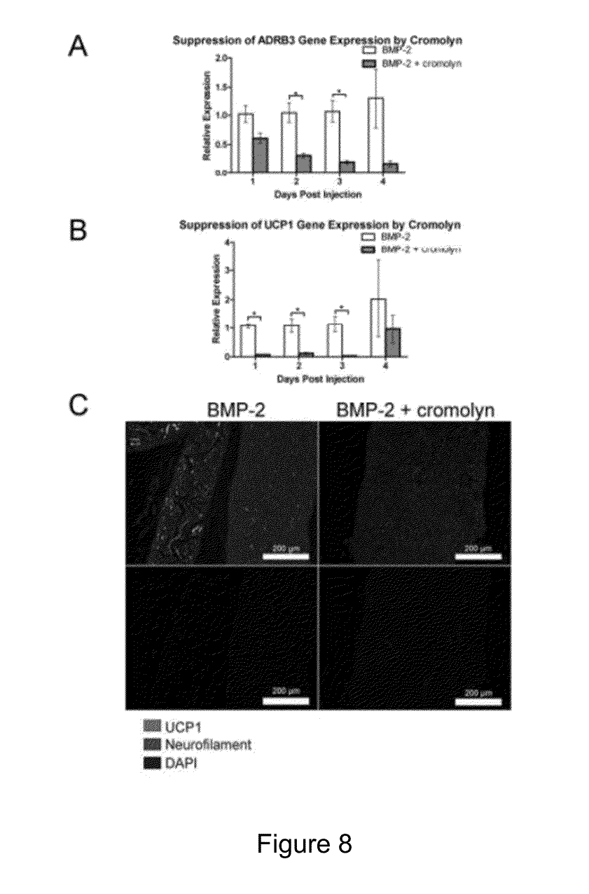

It has been previously demonstrated that BMP2 induces sensory nerve remodeling, through recruitment of mast cell degranulation (Salisbury et al., 2011, J Cell Biochem 112(10):2748-58; Kan et al., 2011, J Cell Biochem 112(10):2759-72) (FIG. 1A). Here it is examined whether mast cell degranulation, leads to the local release of noradrenaline, activation of SNS signaling, and expansion and release of cells from the perineurial region of the peripheral nerves. Thus, blocking mast cell degranulation, should suppress SNS signaling, nerve remodeling and brown adipogenesis. To test this prediction, mast cell degranulation was blocked and changes in ADRB3 and UCP1-specific RNA were examined within the tissues. Animals were pretreated with the drug sodium cromoglycate (cromolyn), which has been shown to prevent mast cell degranulation (Cox, 1967, Nature 216:1328-1329) and reduce heterotopic bone formation (Salisbury et al., 2011, J Cell Biochem 112(10):2748-58). Heterotopic ossification (HO) was then induced in these animals and RNA extracted at specific time points following injection of BMP2 expressing cells. FIG. 8A and FIG. 8B presents RNA expression in cromolyn-treated animals given injections of AdBMP2 transduced cells relative to RNA expression in untreated animals given injections of AdBMP2 transduced cells. A substantial suppression of both ADRB3 and UCP1 (p<0.05) RNA levels was observed in the cromolyn treated tissues, as compared to untreated tissues. The nerves in paraffin embedded tissue sections of the hind limb from cromolyn treated or untreated animals 2 days after induction of HO was also analyzed by immunostaining for UCP1. As seen in FIG. 8C, in animals that were not pre-treated with cromolyn, cells positive for UCP1 were observed associated with the nerve, identified by neurofilament (NF) staining. However, in tissues from animals that received cromolyn, expression of UCP1 associated with the nerve was not observed. This data further suggests this pathway involving both mast cell degranulation and sympathetic activation is important for the production of BALCs.

### BALCs Express Reelin

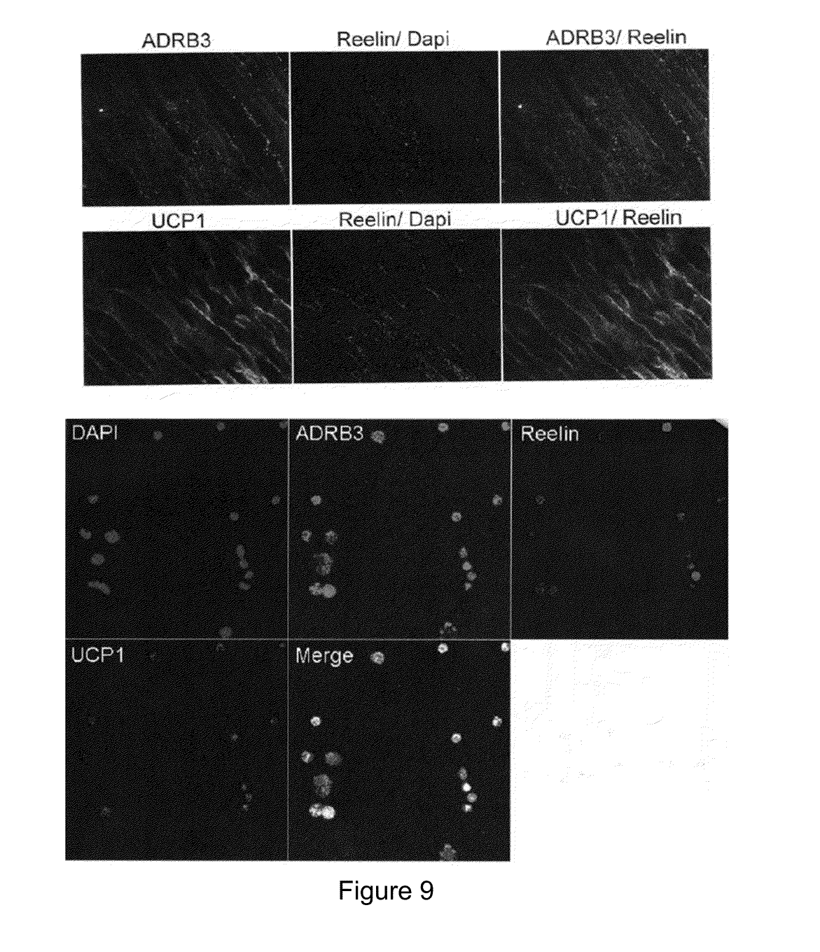

Preliminary experiments indicate that when BMP2 induction is performed in an ApoE−/− mouse that not only is bone not formed, but the orderly pattern of events is dramatically altered. Other preliminary data indicates that BMP2 also initiates neurogenesis in this model. The expression of reelin by BALCs was therefore examined, since reelin and ApoE utilize the same receptors and ApoE interferes with reelin binding (D'Arcangelo et al., 1999, Neuron 24:471-479). Additionally, astrocytes within the brain are closely associated with cerebral vessels and are intimately involved in their patterning. Additionally, these glial cells are known to be induced by BMP2 (Falconer, 1951, Journal of Genetics 50:192-205) the pattern of expression of the astrocyte-specific molecule reelin was therefore analyzed in these cells three days after BMP2 induction. In FIG. 9 (upper panel) co-expression of reelin, UCP1, and ADBR3 is observed at this time point. However, it is difficult to discern if the same cells were expressing all three markers. ADRB3+ cells were therefore isolated by FACS and their expression of each marker was determined by IHC analysis after cytospin. In FIG. 9 (lower panel), it is apparent that ADRB3+ cells co-express UCP1 and reelin.

### Expansion of ADRB3+Cells and Implications

The results presented herein demonstrate the presence of a cell within the perineurial region of peripheral nerves that expands and undergoes migration towards BMP2 expressed within soft tissues. While these cells migrate they undergo brown adipogenesis, with 100% of the ADBR3+ cells isolated in soft tissues, expressing UCP1. This process could be totally ablated by delivery of cromolyn, which prevents mast cell degranulation. In the presence of cromolyn, mast cells would not release stored serotonin preventing expression of noradrenaline. Further, release of mast cell chymase and proteases would prevent remodeling of the nerve extracellular matrix and could also inhibit release of the cells.

The ADBR3+ cells are initially localized in the peripheral nerve. In the current studies it is described that a portion of these precursor cells appear associated with the perineurium of the nerve. The nerve structure has long been characterized to have an internal endoneurial region, consisting of either myelinating or non-myelinating schwann cells that surround axons. The myelinated axons are separated from the rest of the nerve by a myelin sheath, while the perineurium separates the endoneurial and epineurial regions. A strong HNK staining, a marker of migrating neural crest cells, is also present not only in ADRB3+ outside the nerve (FIG. 11), but also in cells immediately adjacent to the axon (FIG. 11) and these cells are not positive for ADRB3. Therefore it is also conceivable that these perineruial adipocyte-like cells arise from more primitive cells in the endoneurium. Although little is known about the embryonic origin of adipocytes, a few studies report that at least some adipocytes originate from neural crest (Billon et al., 2007, Development 134:2283-2292), which is consistent with the results reported in this paper.

It is intriguing that ADRB3 and UCP1 expression in the nerve shows the majority of co-expression in the perineurial region, but also positive ADRB3 expression in other cells, suggesting that ADRB3 populations may be contributing to the replacement of cells. A similar mechanism has been shown in the brain, where neural stem cells in the subventricular zone (SVZ) are activated by noradrenaline and ADRB3 plays a critical role in this activation (Jhaveri et al., 2010, J Neurosci 30:2795-2806). Adipocytes are typically thought to derive from the mesoderm during development, but a recent study demonstrated a subset of adipocytes originating from the neural crest (Billon et al., 2007, Development 134:2283-2292). Perhaps the nerve represents a niche for specialized progenitors that can ultimately be replaced from a neural stem cell like cell. De-differentiated specialized Schwann cells have already been demonstrated to expand and migrate as melanocyte precursors in skin (Adameyko et al., 2009, Cell 139:366-379). The presence of a P75 positive cell, expressing markers of a more primitive stem cell, which also co-express the osteoblast specific transcription factor osterix was recently reported (Salisbury et al., 2011, J Cell Biochem, 112: 2748-2758).

Previous research has established SNS control, via noradrenaline release and adrenergic receptor stimulation, of the proliferation, differentiation, and activity of classical, resident BAT (Cannon and Nedergaard, 2004, Physiol Rev 84:277-359; Klingenspor, 2003, Exp Physiol 88:141-148). A similar mechanism for BALCs from peripheral nerves is demonstrated herein. However, BALCs, interscapular BAT, and BAT derived from white fat have significant differences. While not wishing to be bound by any particular theory, the cells identified herein appear to be transient. The results show rapid expansion of ADBR3+ cells within the perineurial region as well as similarly rapid egress from the nerve, with a simultaneous rapid increase in UCP1 expression both at an RNA and protein level. Intriguingly, this RNA production appears to drop just as rapidly, suggesting that these cells may be present for only a short time, such as during release of BMPs from tissues after injury. These cells thus seem very different from the other kinds of BAT well described in the literature, since the various transcription factors that have been shown to be involved in the biogenesis of both interscapular BAT and the BAT derived from white adipocytes, are absent. Peroxisome proliferator-activated receptor gamma (PPARγ) is regarded as a central regulator of adipogenic differentiation (Rosen et al., 1999, Mol Cell 4:611-617; Nedergaard et al., 2005, Biochim Biophys Acta 1740:293-304), and PRDM16 controls the development of brown adipocytes in traditional BAT depots (Seale et al., 2008, Nature 454:961-967; Seale et al., 2007, Cell Metab 6:38-54; Kajimura et al., 2008, Genes Dev 22:1397-1409), as well as promotion of brown adipocytes induced by adrenergic stimulation within white fat (WAT) depots (Seale et al., 2011, J Clin Invest 121:96-105). Significant changes, in gene expression, of either PPARγ or PRDM16 after BMP2 stimulation was not detected. This may suggest different molecular pathways and sources for the brown adipocytes in BALC biogenesis, as compared to embryonically established BAT depots. Furthermore, PRDM16 promotes a brown fat phenotype within subcutanenous WAT depots (Seale et al., 2011, J Clin Invest 121:96-105), but white adipocytes switching to a brown adipocyte-like phenotype was only identified when heterotopic bone formation is induced in Misty mice, which lack BAT (Olmsted-Davis et al., 2007, Am J Pathol 170:620-632). WAT as a source for brown fat like cells occurred under these circumstances as a compensatory measure, indicating again a different, primary pathway for the rapid generation of brown adipocytes during HO. Further credence is given to the idea that this primary pathway may involve the nerves, as the mutation of Misty mice has now been assigned to Dock 7, a gene related to neuronal function (Blasius et al., 2009, Proc Natl Acad Sci USA 106:2706-2711). Finally, it has been previously noted that PPARγ-ablated brown adipose tissue can express UCP1, and PGC-1α coactivates other transcription factors (including PPARa); thus, the significance of PPARγ for the physiological control of UCP1 gene expression is not settled even in dBAT (Nedergaard et al., 2005, Biochim Biophys Acta 1740:293-304).

It is suggested that the ultimate function of BALCs is regulation of microenvironmental oxygen tension. The ability to lower oxygen tension using uncoupled respiration is critical not only for chondrogenesis (Olmsted-Davis et al., 2007, Am J Pathol 170:620-632), but also enables, by upregulation of HIF1 and secretion of VEGF-D (Dilling et al., 2010, J. Bone Mineral Res., 25: 1147-1156), not only neovascularization (Salisbury et al., 2011, J Cell Biochem 112(10):2748-58) but also, most likely, lymphangiogenesis since this particular form of VEGF is a powerful inducer of lymphoid formation. This may be particularly important for removal of edema, since another important product of the electron transport chain is water. The relationship of adrenergic nerves and lymphatics is also well known since all primary and secondary lymphoid organs in the body are innervated by adrenergic neurons. Therefore it is likely that BALCs controls and guides the formation of a neurovascular unit for ultimate innervations and vascularization of newly formed bone. It ultimately does this by control of microenvironmental oxygen tension on the one hand, and directed synthesis of neural (reelin) and vessel guidance (VEGF-D) molecules. Supporting this contention is the fact that VEGF-D has also recently been shown to be a neural guidance molecule.

One surprising finding is the expression of reelin by BALCs. Reelin was discovered in 1995 as not only being the protein responsible for the Reeler mouse phenotype (Falconer, 1951, Journal of Genetics 50:192-205), but also was found present only in pioneer neurons that guided the formation of complex neural networks (Hirotsune et al., 1995, Nat Genet 10:77-83).

Described herein, it is suggested that BALCs, by virtue of their position, establishes gradients of oxygen tension that determine cell fate. Recently, it has been shown by Hochstim (Stone et al., 1995, J Neurosci 15:4738-4747) that astrocytes establish positional identity due to morphogen gradients, and this positional identity is established by expression of reelin and slit. Additionally, it has also been shown that during retinal development regions of hypoxia, occupied by astrocytes, secrete VEGF causing neovascularization (Besson et al., 2010, Hum Mol Genet 19:3372-3382). In other recent work on Huntington's disease, it has been determined that despite the presence of mutant huntingtin protein in glial cells, an increase in uncoupling proteins could alleviate the Huntingtin's disease phenotype (Motyl and Rosen, 2011, Discov Med 11:179-185). It therefore seems that glial cells, under certain conditions, can regulate oxygen tension in a manner similar to what is described for BALCs. It is therefore suggested that BALCs exhibits similarity to glial cells in the CNS, particularly astrocytes, and indeed may be a transient glial cell that is induced in response to BMP2. This means that such glial cells may play an intimate role in bone formation and even bone homeostasis since the role of brown fat in such homeostasis has recently been suggested (Besson et al., 2010, Hum Mol Genet 19:3372-3382).

It is described herein, for the first time, that peripheral nerves house the progenitors for BALCs. Although BMP2 may be involved in the process of induction of these progenitors, other molecules that are known to be critical such as Dock 7, may participate in the egress of these progenitors from the nerve. It is interesting to speculate on the role that blocking such release may have on a number of diseases including those as diverse as Huntington's disease osteoporosis, breast cancer, pancreatic cancer, neuroblastoma, osteosarcoma, and neurofibromatosis.

### Example 2

### Presence of UCP1⁺ Brown Adipocytes Stem Cells in the Perineurium of Peripheral Nerves

BMP2 can induce neuro-inflammation in dorsal root ganglia cultures and plays a key role in nerve patterning in the embryo. Previous studies suggest that BMP2 asserts direct effects on peripheral nerves in vivo, leading to release of inflammatory mediators substance P and calcitonin gene related protein (CGRP) (Salisbury et al., 2011 Journal of cellular biochemistry 112:2748-2758). BMP2 (approximately 20 ng per day) similar to physiological release of the protein during fracture (Fouletier-Dilling et al., 2007 Hum Gene Ther. 18: 733-745) was delivered by way of delivery of cells transduced with AdBMP2. These AdBMP2-transduced cells survived in the tissue at the site of injection for approximately 6 days (Olmsted-Davis et al., 2002 Human gene therapy 13: 1337-1347; Gugala et al., 2003 Gene therapy 10: 1289-1296). Within 48 hours after delivering BMP2, mast cells within the peripheral nerves adjacent to the injection site underwent degranulation (Salisbury et al., 2011 J Cell Biochem 112:2748-2758) and a coordinated expression of activated MMP9 took place (Rodenberg et al., 2011 Tissue engineering Part A 17: 2487-2496) ultimately leading to remodeling of the matrix of the nerve. Mast cells also released serotonin during degranulation (Wilhelm et al., 2005 The European journal of neuroscience 22: 2238-2248), which bound to the 5-HT receptor and led to the release of noradrenaline, which in turn stimulated β-adrenergic receptor (ADRB) signaling pathways. In the present models, there was a significant elevation in circulating noradrenaline, coincident with sympathetic nervous system (SNS) activation (Salisbury et al., 2012 Stem Cells Transl Med 1(12): 874-85).

It has been observed that this process leads to the rapid replication of the ADRB3⁺ cells within the perineurium. Quantization of ADRB3⁺ cells within the soft tissues by FACS showed a significant increase in the number of these cells between 2 and 4 days after BMP2 induction. Mice receiving cells transduced with the control virus, in all cases, yielded results similar to mice that had no injection, indicating that BMP2, directly or indirectly, leads to expansion of the ADBR3⁺ cells. In support of the notion that these cells are expanding, a significant increase in ADRB3-specific RNA was also observed during this time frame. Alternatively, there was a steep decline in nerve-associated ADRB3⁺ cells at the same time suggesting that the perineurial ADRB3⁺ cells may be migrating from the nerve. In fact, the ADRB3⁺ population within the nerve 4 days after BMP2 induction was significantly lower than at a resting state (Salisbury et al., 2012 Stem Cells Transl Med 1(12): 874-85).

The migration of these cells was confirmed by noting the expression of the carbohydrate moiety HNK1, which has been shown to be essential for neural stem cell migration (Bronner-Fraser, 1986 Developmental biology 115: 44-55; Dottori et al., 2001 Development 128: 4127-4138). Quantization of HNK1 expression on ADRB3⁺ cells revealed a significant (p<0.05) three-fold increase 2 days after delivery of BMP2. Co-expression of uncoupling protein 1 (UCP1) was noted at the same time as these cells migrated from the nerve. After four days of BMP2 induction, ADRB3⁺ cells were isolated by FACS and immunostained for UCP1 expression. Interestingly, 100% of the cells were positive for both markers. UCP1 is widely used as a marker of brown adipose (BAT). Generation of brown adipose tissue has been linked to activation of the SNS (Cannon et al., 2004 Physiol Rev. 84: 277-359; Lowell et al., 2000 Nature 404: 652-660; Klingenspor et al., 2003 Exp Physiol. 88: 141-148; Collins et al., 2010 Int J Obes (Lond) 34 Suppl 1:S28-33) and β₃-agonists have been shown to induce UCP1⁺ BAT-like cells in mice, dogs, and adult humans (Harper et al., 2008 Annu Rev Nutr. 28: 13-33). The appearance of brown adipocyte-like cells during heterotopic ossification (HO) has been previously reported (Olmsted-Davis et al., 2007 The American journal of pathology 170: 620-632) that appeared to direct new vessel formation (Dilling et al., 2010 Journal of bone and mineral research 25:1147-1156) and control oxygen tension (Olmsted-Davis et al., 2007 The American journal of pathology 170: 620-632) within the tissue. The induction of UCP1 in the ADBR3⁺ cells in the presence of BMP2 is supported by the large (70-fold) change in UCP1-specific RNA level. The data suggests that ADRB3⁺ perineurial progenitors expand and undergo BAT-like differentiation. No UCP1 expression was observed in untreated or control mice or in the ADRB3⁻ cell population (Salisbury et al., 2012 Stem Cells Transl Med 1(12): 874-85).

The data collectively suggests that these cells are essential for BMP2 induced bone formation, and may be a critical component to systems that depend on BMP2 for bone healing. Further, they can be readily purified from peripheral nerve through digestion from the peripheral nerve and fluorescent activated cell sorting (FACS) isolation using ADRB3 receptor expressed only on these cells in peripheral nerves. This is the first report that shows the presence of UCP1⁺ brown adipocytes stem cells in the perineurium of peripheral nerves.

### Example 3

### Isolation of Stem Cells from Human Peripheral Nerves

Peripheral nerves are complex multi-layered structures comprising of several cell types. These layers are called the outermost epineurium, inner perineurium and innermost endoneurium. Axons are localized within the endoneurium, forming separate bundles surrounded by the perineurium (or nerve sheath). This structure is known as a fascicle. Human peripheral nerves comprise of several fascicles (as many as 50 in the sciatic nerve). These fascicles are contained within the outermost layer called the epineurium. The epineurium comprises predominantly of longitudinal arrays of collagen fibers that provide structural stability to the entire nerve. Unlike its human counterpart, the mouse sciatic nerve includes predominantly of a single fascicle surrounded by its perineurium and some loose connective tissue. The epineurium is extremely small, and lacks the organized structure of its human counterpart. The perineurium comprises mainly of specialized epitheloid myofibroblasts organized in concentric layers through which epineurial arterioles and post-capillary endoneurial venules transverse. The perineurium and endoneurial microvessels possess restrictive tight junctions. These form the blood-nerve interface, necessary for maintaining peripheral nerve internal homeostasis necessary for normal axonal function (Yosef et al., 2010 Journal of neuropathology and experimental neurology 69: 82-97). During axonal sprouting and growth, perineurial alterations occur in association with Schwann cell proliferation and maturation. Little is known about these perineurial alterations. The adrenergic receptor β3 receptor (ADRB3) was recently identified on the surface of a progenitor within the perineurium of mouse nerve, suggesting that these cells may respond to external stimuli associated with nerve injury and pathogenic remodeling. However, in humans these external stimuli may not be adequate to penetrate the more complex structures significantly reducing the ability of these cells to be able to contribute to tissue regeneration. Without wishing to be bound by any particular theory, it is believed that the massive doses of BMP2 protein required for human bone healing is directly linked to the ability of BMP2 to mobilize these cells in humans. Further, nerve regeneration may also be hampered by the ability of these cells to expand and migrate from the nerve. Experiments were performed to confirm the presence of these cells within the perineurium, confirm that we could isolate these cells, and to determine that they behaved similarly to the UCP1⁺ brown adipocytes.

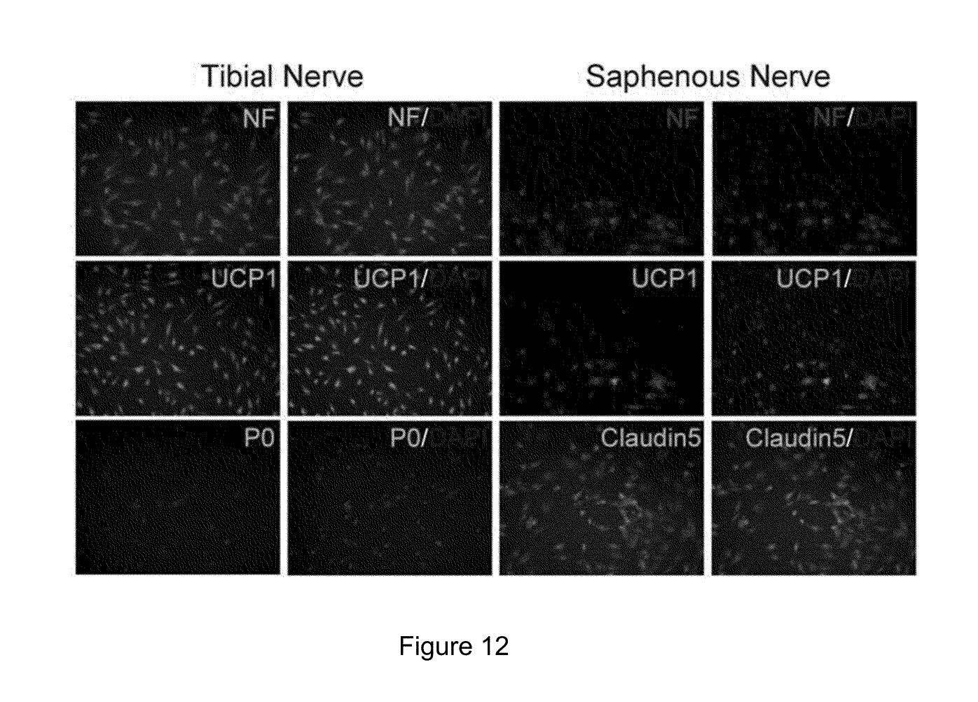

Human peripheral nerves were obtained under an IRB approved protocol. The tissues were obtained fresh and directly plated in culture media (DMEM supplemented with 10% serum) to allow the cells to migrate from the tissues. The nerve tissue was removed from each dish at weekly intervals, and resultant cells remaining in the well labeled as to the dates of tissue removal. Serial culturing was performed for 4 weeks, and then expanded cells were isolated and frozen for later use in animals experiments, or immunostained for various markers to confirm there phenotype. Significant variation in the phenotype of these populations was observed. FIG. 12 shows the initial cells to expand and migrate from the nerve from two different types of nerves (one being only sensory the other sensory and motor).

Immunohistochemical analysis of these cells showed that they expressed neurofilament H, a marker of the neuron-axon itself. This was strikingly obvious in the cultures from the saphenous nerve, which appeared to be somewhat mixed in that only one half of the culture expressed NF, whereas all the cells from the tibial nerve expressed this marker. This was surprising since this suggests that these cells may be more pluripotent, initially expressing a neurite-axonal marker. These cells surprisingly also appeared to be associated with the UCP1 expression. The UCP1 was less robust in the saphenous populations that had elevated NF expression, whereas in the tibial nerve the cells were predominantly UCP1⁺ but had much lower levels of NF. Both populations were negative for the Schwann cell marker P0. The population of cells in the tibial nerve was negative for Claudin 5 a marker associated with endoneurial endothelial cells, and although present in the saphenous nerve mixed population, it did not align with the UCP1 positive cells suggesting that these are different populations. Interestingly, the data suggests that ADRB3⁺ cells can be isolate and cultured, yet retaining the UCP1⁺ expression.

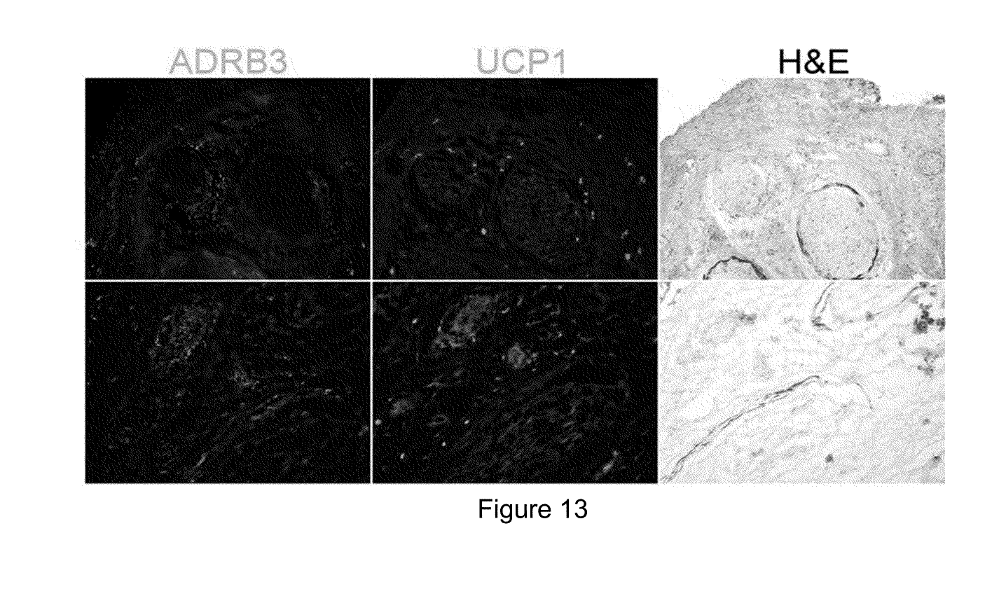

Experiments were also performed to characterize the cells in vivo within tissue sections generated from the human peripheral nerves. As shown in FIG. 13, the immunohistochemical staining for ADRB3 showed a significant number of positive cells within the perineurial region of the nerve. Interestingly, this nerve cross section taken from nerve isolated during amputation of a limb, from a diabetic patient, appears to show several intact fascicles as indicated by the circular line (perineurium) whereas one of the fasicles is no longer organized and there are a large number of what appear to be replicating ADRB3⁺ cells within the nerve with a subset of these expressing UCP1. The UCP1 expression was considerably fewer numbers than the culture, again suggesting that either the UCP1⁺ cells expanded or that the stem cells went on to differentiate into brown adipocytes. The data suggests that not only are the brown adipose progenitors present in the human nerve, but that in diseases where metabolism is derailed, they may start to replicate and undergo brown adipogenesis, resulting in the loss of the perineurium, and ultimately breakdown in axonal function.

The results presented herein demonstrate that there is a unique stem cell within the perineurium that appears to be able to undergo brown adipogenesis, and is necessary for BMP2 induced bone formation. Further, these cells can be isolated and readily expanded in culture.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. An isolated perineurium derived adult stem cell capable of differentiating into brown adipose tissue.

2. The cell of claim 1, wherein the cell expresses β3 adrenergic receptor (ADRB3).

3. The cell of claim 1, wherein the cell expands in response to stimulation with BMP-2.

4. A brown adipose tissue like cell derived from the cell of claim 1.

5. The cell of claim 4, wherein the cell expresses UCP-1.

6. An astrocyte like cell derived from the cell of claim 1.

7. The cell of claim 6, wherein the cell expresses reelin.

8. The cell of claim 1, wherein the cell is pluripotent.

9. The cell of claim 8, wherein the cell retains the ability to differentiate into a germ layer selected from the group consisting of mesoderm, ectoderm, endoderm, and any combination thereof.

10. The cell of claim 1, wherein the cell is isolated from the perineurium of a peripheral nerve.

11. A method of generating an isolated population of perineurium derived adult stem cells, the method comprising isolating a peripheral nerve from a subject and extracting cells from the perineurium of the peripheral nerve.

12. The method of claim 11, further comprising separating the extracted cells by selecting for cells expressing ADRB3.

13. The method of claim 11, further comprising culturing the extracted cells.

14. A method of promoting bone growth, the method comprising administering a population of perineurium derived adult stem cells to a region in need of bone growth in a subject.

15. The method of claim 14, further comprising administering BMP-2 to the region.

16. The method of claim 14, wherein the perineurium derived adult stem cells are present within a biocompatible scaffold.

17. The method of claim 16, wherein the biocompatible scaffold comprises perineurium derived adult stem cells and osteoblasts.

18. The method of claim 16, wherein the biocompatible scaffold comprises perineurium derived adult stem cells and osteoprogenitor cells.

19. A method of promoting neuroregeneration, the method comprising administering a population of perineurium derived adult stem cells to a region in need of neuroregeneration in a subject.

20. The method of claim 19, further comprising administering BMP-2 to the region.

21. The method of claim 19, wherein the perineurium derived adult stem cells are present within a biocompatible scaffold.

22. The method of claim 21, wherein the biocompatible scaffold comprises perineurium derived adult stem cells and neural progenitor cells.
